(19) United States
(12) Patent Application Publication
(10) Pub. No.: US 2003/0144190 A1
(43) Pub. Date: Jul. 31, 2003

(54) Method of modulating and antiapoptotic and antiapoptotic pathways in cells

(76) Inventors: Nahum Sonenberg, Quebec (CA); Anne-Claude Gingras, Quebec (CA); Vitaly A. Polunavsky, Plymouth, MN (US); Peter B. Bitterman, Edina, MN (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/148,599

(22) PCT Filed: Dec. 1, 2000
(86) PCT No.: PCT/CA00/01465

(51) Int. Cl. A61K 38/17

(52) U.S. Cl. 514/12

(57) ABSTRACT

More than 30% of human malignancies harbor encogenic Ras. Both pro-apoptotic and anti-apoptotic pathways emanate from encogenic Ras with survival being dominant. Ras survival signaling is thought to be controlled by transcriptional and post-translational processes. The present invention shows that a repressor of cap-dependent translation initiation, 4E-BP1, selectively activates apoptosis in Ras-transformed fibroblasts and eliminates Ras-induced chemoresistance. These effects of 4E-BP1 are strictly dependent on its ability to sequester translation initiation factor eIF4E, thereby preventing its assembly into an active pre-initiation complex. These results suggest that translational control is critical for prevention of apoptosis and resistance to antitumor agents in Ras-transformed cells.

### FIELD OF THE INVENTION

Generally, the present invention relates to methods of modulating proapoptotic and antiapoptotic pathways in cells displaying an enhancement of translation, and in particular, cap-dependent or eIF-4E-dependent translation. In particular, the invention relates to methods of modulating the apoptotic pathways in Ras-transformed cells. In addition, the invention relates to fast growing cells and more particularly to cancer. More specifically, the invention relates to Ras-induced malignancies. The invention also relates to proapoptotic and antiapoptotic pathways emanating from oncogenic Ras and converging on FRAP. The invention further relates to methods of reversing Ras-induced chemoresistance of cancer cells. Conversely, the invention relates to the induction of a blockade of apoptosis leading to chemoresistance of cells. The invention relates to methods of treating cancer as well as to assays and method to identify agents that can modulate the proapoptotic and antiapoptotic pathways dependent on an oncogenic enhancement of translation. In addition, the invention relates to apoptosis modulation in fast growing cells and especially to the induction and/or stimulation of the proapoptotic pathway in fast growing cells.

### BACKGROUND OF THE INVENTION

Extracellular survival factors suppress the intrinsic apoptotic apparatus through cognate receptor kinases at the cell surface which activate the proto-oncogene ras, and a plethora of Ras-induced effector pathways (1). The net effect of activated Ras on cell viability is determined by the balance of its downstream effectors, which can either maintain viability or lead to apoptosis (2). In other words, oncogenic Ras is a cornerstone factor whose effect on cell viability is determined by the relative balance of downstream effectors which stimulate or restrain Ras-induced signalling pathways depending upon the cellular context.

One mediator of Ras survival signaling is phosphatidylinositol 3-kinase (Pl3K) (2, 3), which activates at least two effectors previously implicated in the suppression of apoptosis, transcription factor NF-KB (4) and the serine/threonine protein kinase, Akt (2, 5). Akt plays a key role in transducing receptor-activated survival signaling. Akt-mediated phosphorylation of the Bcl-2 family member Bad and the cell death protease caspase-9 suppresses the intrinsic apoptotic machinery (6), whereas phosphorylation of the Forkhead family transcription factor FKHRL1 by Akt prevents expression of cell death genes such as Fas ligand (7).

Another putative downstream effector of Akt is the FRAP/mTOR kinase (FRAP) which functions in the control of translation by phosphorylating the ribosomal protein kinase p70^{S6K} and the translational repressor 4E-BP1 (also designated PHAS-1) (5, 8). The role of FRAP and its effector pathways in the regulation of apoptosis is incompletely understood. Although the FRAP inhibitor rapamycin apparently does not block growth factor survival signaling (2, 9), it does prevent Ras blockade of apoptosis in growth factor deprived cells (10), and chemosensitizes cancer cell lines expressing activated Ras, even under growth factor replete conditions (11).

A further potential Ras effector molecule is translation initiation factor eIF4E (14), the mRNA cap binding protein which is essential for initiation of cap dependent translation (15). Overexpressed eIF4E causes malignant transformation of immortalized cells (16) and cooperates with Myc and E1A in transformation of primary fibroblasts (17). At least in part, the oncogenic activity of eIF4E may result from its anti-apoptotic function, since ectopically expressed eIF4E rescues growth factor restricted fibroblasts from both Myc-dependent and Myc-independent apoptosis (18). Function of eIF4E is inhibited by the members of a family of translational repressors, the 4E-binding proteins (4E-BPs, also known as PHAS) (19). When hypophosphorylated, 4E-BPs compete with eIF4G for binding with eIF4E and sequester eIF4E in a non-functional complex. Upon hyperphosphorylation in response to extracellular stimuli, 4E-BPs dissociates from the complex with eIF4E. Unsequestered eIF4E binds to eIF4G, forming an active translation initiation complex (19, 20). When ectopically expressed in src-transformed or eIF4E-transformed NIH 3T3 cells, 4E-BP1 inhibits cell proliferation, reverts the transformed phenotype, and suppresses tumorigenicity in vivo (21). In recent years, the pathway leading to phosphorylation of 4E-BPs has been elucidated: it consists of the PI3K/Akt/FRAP kinase cascade in which activated FRAP directly phosphorylates 4E-BPs in a rapamycin-sensitive manner, establishing a link between rapamycin and the function of 4E-BPs (22). The implication of the interaction between eIF4E and 4E-BP1 and between eIF4E and 4E-BP2 have been described in U.S. Pat. No. 5,874,231. More particularly, U.S. Pat. No. 5,874,231 teaches methods for identifying agents that mimic the activity of a hormone in modulating the eIF4E-[4E-BP1] and eIF4E-[4E-BP2] interactions. A number of assays and methods are described therein.

There thus remains a need to better define the factors involved downstream of Ras which contribute directly or indirectly to the proapoptotic and antiapoptotic pathways emanating from oncogenic Ras. Thus, while oncogenic Ras' implication in the proapoptotic and antiapoptotic pathways has been amply demonstrated, the role of downstream effectors of oncogenic Ras converging on translation initiation have merely been proposed. In addition, there remains a need to formally assess whether the oncogenic Ras-induced antiapoptotic and proapoptotic pathways involve translation and more particularly translation initiation. There also remains a need to provide agents and methods to modulate the proapoptotic and antiapoptotic pathways downstream of oncogenic Ras. More particularly, there remains a need to provide a method for preventing Ras blockade of apoptosis, to chemosensitize cancer cell lines expressing activated Ras, and agents therefor.

The present invention seeks to meet these and other needs.

The present description refers to a number of documents, the content of which is herein incorporated by reference, in their entirety.

### SUMMARY OF THE INVENTION

The present invention broadly concerns the identification of translation (and more particularly of translation initiation) as a critical biochemical process (in addition to the transcriptional and post-translational mechanisms) regulating cell viability downstream of an oncogenic activation of translation. The invention also broadly concerns an induction of apoptosis in fast growing cells and especially in cancer cells. In a particularly preferred embodiment of the present invention, there is provided an induction of the proapoptotic pathway in Ras-transformed cells comprising a sequestering of eIF4E.

In addition, the present invention broadly concerns the formal determination of cap-dependent translation as a downstream effector of a transformation phenotype associated with an oncogene-induced enhancement of cap-dependent translation. In a particular embodiment, the present invention pertains to the identification of cap-dependent translation as a downstream effector of oncogenic Ras, and more particularly of 4E-BP1 (a model eIF-4E-sequestering agent).

The invention further relates to a modulation of the relative balance of downstream effectors of Ras, thereby shifting the pathway towards apoptosis or cell survival.

In one preferred embodiment, the present invention concerns a method of activating apoptosis in a transformed cell, thereby eliminating an oncogene-induced chemoresistance. In a particular embodiment, the cell is a Ras-transformed cell and the method enables an elimination of Ras-induced chemoresistance.

In another embodiment, the present invention relates to a method of immortalizing a transformed cell. In a non-limiting example, this transformed cell is a Ras-transformed cell.

The present invention in addition relates to a method of reverting a transformed cell displaying an oncogene-dependent enhancement of translation to a non-malignant phenotype, by diminishing or inhibiting cap-dependent translation. In one embodiment, the method comprises a reversion of a Ras-transformed cell to a non-malignant phenotype. In a preferred embodiment, the invention relates to a method to selectively induce cell death (apoptosis) in Ras-transformed cells.

According to one aspect of the present invention there is provided a method of sensitizing an oncogenic Ras-transformed cell to apoptosis comprising inhibiting the translation, through a downstream effector of Ras herein identified, translation initiation factor eIF4E. In a particularly preferred embodiment of the present invention, translation inhibition through eIF4E is effected by the translation inhibitor 4E-BP1. In one embodiment, the invention thus broadly concerns a modulation of the Ras-induced apoptotic pathway through a modulation of eIF4E-dependent translation. The invention thus broadly concerns a modulation of the Ras-induced apoptotic pathway through a modulation of eIF4E-dependent translation. In one particular embodiment, such a modulation of eIF-4E-dependent translation is effected by a repressor of cap-dependent translation initiation, 4E-BP1, which is shown to selectively activate apoptosis in Ras-transformed fibroblasts and eliminate Ras-induced chemoresistance. These effects of 4E-BP1 are strictly dependent on its ability to sequester translation initiation factor eIF4E, thereby preventing its assembly into an active pre-initiation complex with eIFGI.

According to another aspect of the invention the molecular target of FRAP kinase conferring Ras-induced viability and chemoresistance was identified. Hence, the instant inventors identified a novel survival pathway from Ras through FRAP to 4E-BP1-inhibitable translation initiation, providing new insights into the biology of cancer.

According to a further aspect of the present invention it was found that ectopic expression of 4E-BP1 selectively kills and chemosensitizes Ras-transformed cells having clear implications for cancer therapeutics. Many properties of eIF4E and 4E-BP1, including their ability to regulate proliferation, apoptosis and drug resistance make them potential therapeutic targets in human malignancy. This is further exemplified using naturally occurring cancer cells.

The invention also relates to assays and methods for screening and identifying agents which can modulate apoptosis in a cell through a modulation of eIF-4E-dependent translation. For certainty, the identification of eIF-4E and more particularly 4E-BP1 as targets for modulating apoptosis in Ras-transformed cells provide important and broad modalities to treat malignancy or identify therapeutic agents.

Having now identified and validated the interaction between 4E-BP1 and eIF4E as a target for apoptosis modulation opens the way to the identification of further targets in the same pathway (i.e. translation control). Non-limiting examples of such targets include eIF4F, kinases, phosphatases or other agents affecting the 4E-BP1-eIF4E interaction, or affecting the activity and/or the level of eIF4E. Thus, while in one preferred embodiment of the present invention in which induction of apoptotis is desired, the reduction and/or inhibition of eIF-4E-dependent translation is effected by 4E-BP1, the instant invention should not be so limited. Indeed, the person of ordinary skill to which the present invention pertains could use other agents to achieve this goal (e.g. other eIF-4E sequestering agents, eIF-4E level-reducing agents, eIF-4E activity-reducing agents . . . ).

In addition, having shown that the apoptotic pathway can be modulated in transformed cells displaying an oncogene-dependent enhancement of translation by modulating eIF-4E-dependent translation has broad implications on the modulation of the apoptotic pathway in cells in which the apoptotic pathway is perturbed by an enhancement of translation initiation and more particularly of eIF-4E-dependent translation. In a preferred embodiment, this modulation of apoptosis is effected in cells which are transformed.

For the purpose of the present invention, the following abbreviations and terms are defined below.

### DEFINITIONS

The terminology “eIF4E sequestering agent” refers to an agent which interacts with eIF4E in a manner such that it reduces or abrogates the translation of eIF4E dependent mRNAs. For example, by inhibiting or reducing eIF4E interaction with eIF4G to constitute eIF4F. It will be understood that an EIF4E desequestering agent has the opposite effect (i.e. it promotes an increase in eIF4E-dependent translation).

The terminology “eIF4E interacting agent” or the like refers to an agent which interacts with eIF-4E in a manner that it can influence or modulate cap-dependent translation. Non-limiting examples of such eIF4E interacting agents include proteins or fragments thereof (e.g., translation factors, enzymes, and kinases), nucleic acids (e.g. mRNA), chemical entities (e.g., m⁷GTP, and m⁷GDP), antibodies and the like.

The terminology “translation factor”, as commonly known in the art, is meant to refer to a group of factors or molecules participating directly in the translation of mRNA into polypeptides. Non-limiting examples thereof include eIF1, eIF2, eIF3 and eIF4A, eIF4B, eIF4E, eIF4F, and eIF4G.

The terminology “modulation of two factors” is meant to refer to a change in the affinity, strength, rate and the like between such two factors. The terminology “modulation of translation” refers to change in the efficiency or rate of translation of mRNAs resulting in a quantitative or qualitative change or rate of protein synthesis.

The terminology “eIF4E-dependent translation” is meant to refer to translation of an mRNA which requires eIF4E for its initiation of translation. As commonly known in the art, different mRNAs show different degrees of dependency on eIF4E for initiation of translation. The presence of the cap structure, consisting of a 7-methylguanasine residue linked to the 5′ position of eukaryotic mRNAs, and the degree of secondary structure between the cap structure and the initiator AUG, are two non-limiting factors which influence the dependency of an mRNA to eIF4E.

The terminology “eIF4E screening assays” or the like, is meant to cover screening assays for compounds which modulate the level of eIF-4E (at the protein or mRNA level) or the activity thereof (directly or indirectly). In view of the fact that (1) eIF-4E is one, if not the cornerstone initiation factor for cap-dependent translation; (2) eIF-4E is significantly regulated; and (3) eIF-4E interacts with a significant number of molecules such as proteins and nucleic acids, the person of ordinary skill will understand that numerous assays can be designed to screen and identify agents which can modulate (e.g. stimulate or inhibit) the apoptotic pathway in cells through a modulation of cap-dependent translation.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, New York).

The present description refers to a number of routinely used recombinant DNA (rDNA) technology terms. Nevertheless, definitions of selected examples of such rDNA terms are provided for clarity and consistency.

As used herein, “nucleic acid molecule”, refers to a polymer of nucleotides. Non-limiting examples thereof include DNA (i.e. genomic DNA, cDNA) and RNA molecules (i.e. mRNA). The nucleic acid molecule can be obtained by cloning techniques or synthesized. DNA can be double-stranded or single-stranded (coding strand or non-coding strand [antisense]).

The term “recombinant DNA” as known in the art refers to a DNA molecule resulting from the joining of DNA segments. This is often referred to as genetic engineering.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (i.e. DNA, RNA or chimeras thereof) for practising the present invention may be obtained according to well known methods.

As used herein, the term “physiologically relevant” is meant to describe interactions which can modulate, for example, initiation of translation of a mRNA in its natural setting.

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 molecules, and they may be adapted to be especially suited to a chosen nucleic acid amplification system. As commonly known in the art, the oligonucleotide probes and primers can be designed by taking into consideration the melting point of hydrizidation thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “DNA” molecule or sequence (as well as sometimes the term “oligonucleotide”) refers to a molecule comprised of the deoxyribonucleotides adenine (A), guanine (G), thymine (T) and/or cytosine (C), often in a double-stranded form, and comprises or includes a “regulatory element” according to the present invention, as the term is defined herein. The term “oligonucleotide” or “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction. Of course, as very well-known in the art, DNA can also be in a single-stranded form.

“Nucleic acid hybridization” refers generally to the hybridization of two single-stranded nucleic acid molecules having complementary base sequences, which under appropriate conditions will form a thermodynamically favoured double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labelled probe in a solution containing 50% formamide, high salt (5×SSC or 5×SSPE), 5×Denhardt's solution, 1% SDS, and 100 μg/ml denatured carrier DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al.,1989, supra).

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988, Ann. Reports Med. Chem. 23:295 and Moran et al., 1987, Nucleic acid molecule. Acids Res., 14:5019. Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Although less preferred, labelled proteins could also be used to detect a particular nucleic acid sequence to which it binds. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation. Probes can be labelled according to numerous well known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (i.e. uniformly labelled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthetised chemically or derived by cloning according to well known methods. Oligos can be single-stranded or double-stranded.

As used herein, a “primer” defines an oligonucleotide which is capable of annealing to a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra) Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. Patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analysed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise the a specific polypeptide or protein. It will be readily recognized by the person of ordinary skill, that the nucleic acid sequence of the present invention can be incorporated into anyone of numerous established kit formats which are well known in the art.

A “heterologous” (i.e. a heterologous gene) region of a DNA molecule is a subsegment segment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (i.e. SDS gel electrophoresis, gel filtration, centrifugation, ion exchange chromatography . . . ). In addition, the protein of interest can be purified via affinity chromatography using polyclonal or monoclonal antibodies. Non-limiting examples of affinity purification include eIF4E purification using a m7GDP column (Edery et al., 1988, Gene 74:517-525) and 4E-BP1 purification using a eIF4E column. The purified protein can be used for therapeutic applications.

The DNA construct can be a vector comprising a promoter that is operably linked to an oligonucleotide sequence of the present invention, which can in turn, be operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain −10 and −35 consensus sequences, which serve to initiate transcription and the transcript products contain Shine-Dalgamo sequences, which serve as ribosome binding sequences during translation initiation.

As used herein, the designation “functional derivative” denotes, in the context of a functional derivative of a sequence whether an nucleic acid or amino acid sequence, a molecule that retains a biological activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid as chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention. Conservative, non-conservative, silent substitutions of amino acids and the like are well-known to the skilled artisan to which the present invention pertains.

Thus, the term “variant” refers herein to a protein or nucleic acid molecule which is substantially similar in structure and biological activity to the protein or nucleic acid of the present invention.

For certainty, although exemplified with 4E-BP1, it should be clear to the skilled artisan that the present invention should not be so limited. For inducing the proapoptotic pathway, a number of means to sequester eIF4E are available. Non-limiting examples of proteins or fragments thereof which could be used to sequester eIF4E include proteins or amino acid sequences comprising an eIF4E binding site. Examples of such proteins include 4E-BP1, 4E-BP2, 4E-BP3 and eIF4G. In addition, in view of the conservation of the eIF4E binding domain of such proteins during evolution, numerous sequences can be synthesized or derived from diverse animal and plant sources.

Similarly, although the present invention is exemplified with Ras-transformed cells or necessarily with transformed cells, it should be clear to the skilled artisan that the present invention should not be so limited. Indeed, the modulation of apoptosis in accordance with the present invention could be effected in non-Ras-transformed cells, provided that the modulation of apoptosis is effected in a cell displaying an apoptotic pathway which is perturbed by a perturbation of eIF-4E-dependent translation (whether enhanced or lowered).

The functional derivatives of the present invention can be synthesized chemically or produced through recombinant DNA technology. All these methods are well known in the art. In one particular embodiment of the present invention, a variant according to the present invention includes an eIF4E sequestering agent, such as a 4E-BP1 variant or fragment which retains its ability in sequestering eIF4E, thereby modulating translation initiation and consequently the apoptotic pathway in a Ras-transformed cell. The interaction domains of eIF4E and 4E-BP1 being known, it is thus possible for the skilled artisan to identify and/or design 4E-BP1 variants having a modified affinity for eIF4E (see the alignments below). In addition, having identified eIF4E-dependent translation as a key biochemical process involved in apoptosis, the present invention provides the means to influence the pro- and anti-apoptotic pathway by modifying the domain of eIF4E which interacts with different sequestering agents, or using agents which target eIF4E or other factors with which it interacts so that a modulation of eIF4E interactions with different initiation factors can occur.

As used herein, “chemical derivatives” is meant to cover additional chemical moieties not normally part of the subject matter of the invention. Such moieties could affect the physico-chemical characteristic of the derivative (i.e. solubility, absorption, half life and the like, decrease of toxicity). Such moieties are examplified in Remington's Pharmaceutical Sciences (1980). Methods of coupling these chemical-physical moieties to a polypeptide are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutations of nucleic acid molecule is a mutant nucleic acid molecule. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in all other cellular components.

As used herein, “eIF-4E biological activity” refers to any detectable biological activity of eIF-4E. This includes any physiological function attributable to eIF-4E. It can include the specific biological activity of eIF-4E in cap-dependent translation initiation. This includes measurement of cap-dependent translation such as, but not limited to: 1) in vivo labeling methods, 2) in vitro translations of mRNAs; and 3) transformation assays or reversal thereof. Monocisronic or bicistronic messages can be used to assess cap-dependent translation, as commonly known (Pelletier et al., 1985). Non-limiting examples of measurements of eIF-4E biological activities may be made directly or indirectly, such as through the detection of a product whose translation is cap-dependent. eIF-4E biological activity is not limited, however, to these biological activities herein identified. Biological activities may also include simple binding or pKa analysis of eIF-4E with the cap structure, kinases, interacting proteins (e.g. translation initiation factors), and the like. For example, by measuring the effect of a test compound on its ability to increase or inhibit eIF-4E binding or interaction is measuring a biological activity of eIF-4E according to this invention. eIF-4E biological activity includes any standard biochemical measurement of eIF-4E such as conformational changes, phosphorylation status or any other feature of the protein that can be measured with techniques known in the art.

In accordance with the present invention, there is also provided a method for identifying, from a library of compounds, a compound with therapeutic effect on disorders implicating a perturbation of the apoptotic pathway. In one particular of the present invention, the disorder implicates highly proliferating cells (e.g. cancer), and the method comprises providing a screening assay comprising a measurable biological activity of eIF-4E; contacting the screening assay with a test compound; and detecting if the test compound modulates the biological activity of eIF-4E; wherein a test compound which modulates the biological activity is a compound with this therapeutic effect.

Also provided within the present invention is a compound having therapeutic effect on disorders implicating a perturbation of the apoptotic pathway, identified by a method comprising: providing a screening assay comprising a measurable biological activity of eIF-4E; contacting the screening assay with a test compound; and detecting if the test compound modulates the biological activity of eIF-4E, wherein a test compound which modulates the biological activity is a compound with this therapeutic effect.

Of course, the method for identifying compounds with therapeutic effect on disorders implicating a perturbation of the apoptotic pathway could involve a disorder based on an enhanced pathway. Non-limiting examples of the disorders for which the present invention finds utility, include inflammatory dieases (e.g. arthritis), cancer, diabetes and obesity.

As used herein, the terms “molecule”, “compound” or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. Non limiting examples of molecules include nucleic acid molecules, peptides, antibodies, carbohydrates and pharmaceutical agents. The agents can be selected and screened by a variety of means including random screening, rational selection and by rational design using for example protein or ligand modelling methods such as computer modelling. The terms “rationally selected” or “rationally designed” are meant to define compounds which have been chosen based on the configuration of the interaction domains of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modelling as mentioned above. Similarly, in a preferred embodiment, the polypeptides of the present invention are modified to enhance their stability. It should be understood that in most cases this modification should not alter the biological activity of the interaction domain. The molecules identified in accordance with the teachings of the present invention have a therapeutic value in diseases or conditions in which the physiology or homeostasis of the cell and/or tissue is compromised by a direct or indirect defect in translation initiation through eIF4E. Alternatively, the molecules identified in accordance with the teachings of the present invention find utility in the development of more efficient molecules which can modulate the eIF4E-[eIF4E sequestering agent] (e.g. eIF4E-[4E-BP1]) interaction.

As used herein, agonists and antagonists of eIF4E-[eIF4E sequestering agent] interaction (e.g. eIF4E-[4E-BP1] interaction) also include potentiators of known compounds with such agonist or antagonist properties. In one embodiment, agonists can be detected by contacting the indicator cell with a compound or mixture or library of molecules for a fixed period of time is then determined.

In one embodiment, the level of gene expression of the reporter gene (e.g. the level of luciferase, or β-gal, produced) within the treated cells can be compared to that of the reporter gene in the absence of the molecules(s). The difference between the levels of gene expression indicates whether the molecule(s) of interest agonizes the aforementioned interaction. The magnitude of the level of reporter gene product expressed (treated vs. untreated cells) provides a relative indication of the strength of that molecule(s) as an agonist. The same type of approach can also be used in the presence of an antagonist(s).

The present invention also provides antisense nucleic acid molecules which can be used for example to decrease or abrogate the expression of a nucleic acid sequence or protein of the present invention (e.g. eIF4E). An antisense nucleic acid molecule according to the present invention refers to a molecule capable of forming a stable duplex or triplex with a portion of its targeted nucleic acid sequence (DNA or RNA). In one particular embodiment, the antisense is specific to 4E-BP1. The use of antisense nucleic acid molecules and the design and modification of such molecules is well known in the art as described for example in WO 96/32966, WO 96/11266, WO 94/15646, WO 93/08845 and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility bu using nucleotide analogs and/or substituting chosen chemical fragments thereof, as commonly known in the art.

Alternatively, an indicator cell in accordance with the present invention can be used to identify antagonists of the 4E-BP1-eIF4E interaction. For example, the test molecule or molecules are incubated with the host cell in conjunction with one or more agonists held at a fixed concentration. An indication and relative strength of the antagonistic properties of the molecule(s) can be provided by comparing the level of gene expression in the indicator cell in the presence of the agonist, in the absence of test molecules vs in the presence thereof. Of course, the antagonistic effect of a molecule can also be determined in the absence of agonist, simply by comparing the level of expression of the reporter gene product in the presence and absence of the test molecule(s).

It shall be understood that the “in vivo” experimental model can also be used to carry out an “in vitro” assay. For example, cellular extracts from the indicator cells can be prepared and used in “in vitro” tests (e.g. binding assays and translation assays).

As used herein, the recitation “indicator cells” refers to cells that express in one particular embodiment an eIF4E sequestering agent and eIF4E, or domains thereof which interact, and wherein an interaction between these proteins or domains thereof is coupled to an identifiable or selectable phenotype or characteristic such that it provides an assessment of the interaction between same. Such indicator cells can be used in the screening assays of the present invention. In certain embodiments, the indicator cells have been engineered so as to express a chosen derivative, fragment, homolog, or mutant of these interacting domains. The cells can be yeast cells or higher eukaryotic cells such as mammalian cells (WO 96/41169). In one particular embodiment, the indicator cell is a yeast cell harboring vectors enabling the use of the two hybrid system technology, as well known in the art (Ausubel et al., 1994, supra) and can be used to test a compound or a library thereof. In one embodiment, a reporter gene encoding a selectable marker or an assayable protein can be operably linked to a control element such that expression of the selectable marker or assayable protein is dependent on the interaction of the eIF4E and 4E-BP1 interacting domains. Such an indicator cell could be used to rapidly screen at high-throughput a vast array of test molecules. In a particular embodiment, the reporter gene is luciferase or β-Gal. In another embodiment, the modulation of 4E-BP1-eIF4E interaction could be assessed by determining the level of translation of specific mRNAs dependent on eIF4E function (e.g. structure of mRNAs, bicistronic mRNAs . . . ). Non-limiting examples of mRNAs which could be used in such assays are known in the art (also see Pelletier et al., 1985, Cell 40:515-526; and ibid, 1988, Nature 334:320-325).

In one embodiment the present invention provides screening assays using eIF-4E, and/or an eIF-4E-sequestering agent which can identify compounds which have therapeutic benefit in disorders in which a perturbation of the apoptotic pathway is encountered. This invention also claims those compounds, the use of these compounds in such disorders, and any use of any compounds identified using such a screening assay in treating such disorders.

Generally, high throughput screens for one or more eIF-4E-dependent translation (herein collectively called cap-dependent translation) modulators i.e. candidate or test compounds or agents (e.g., peptides, peptidomimetics, small molecules or other drugs) may be based on assays which measure biological activity of eIF-4E. The invention therefore provides a method (also referred to herein as a “screening assay”) for identifying modulators, which have a stimulatory or inhibitory effect on, for example, eIF-4E biological activity or expression, or which bind to or interact with eIF-4E, or which have a stimulatory or inhibitory effect on, for example, the expression or activity of eIF-4E interacting proteins (targets) or substrates.

In one embodiment, the invention provides assays for screening candidate or test compounds which interact with substrates of an eIF-4E or biologically active portion thereof (non-limiting examples of such proteins are identified herein).

In another embodiment, the invention provides assays for screening candidate or test compounds which bind to or modulate the activity of an eIF-4E or polypeptide or biologically active portion thereof.

In one embodiment, an assay is a cell-based assay in which a cell which expresses an eIF-4E or biologically active portion thereof, either natural or recombinant in origin, is contacted with a test compound and the ability of the test compound to modulate eIF-4E biological activity (e.g., cap-dependent translation) is determined.

Determining the ability of the test compound to modulate binding of eIF-4E to a substrate, eIF-4E target, or eIF-4E-interacting protein can be accomplished, for example, by coupling eIF-4E, eIF-4 substrate, eIF-4E target, or eIF-4E-interacting protein with a radioisotope or enzymatic label such that binding of the eIF-4E substrate, eIF-4E target, or eIF-4E-interacting protein to eIF-4E can be determined by detecting the label in the eIF-4E-containing complex. For example, compounds (e.g., the targets or substrates) can be labeled with ¹²⁵I, ³⁵S, ¹⁴C, ³²P, or ³H, either directly or indirectly, and the radioisotope detected by direct counting radio-emission or by scintillation counting. Alternatively, compounds can be enzymatically labeled with, for example, horseradish peroxidase or alkaline phosphatase. In these assays, compounds which inhibit or increase eIF-4E substrate-, eIF-4E target- (or the like) binding to eIF-4E are useful for the therapeutic objectives of the invention.

In yet another embodiment, an assay of the present invention is a cell-free assay in which an eIF-4E or biologically active portion thereof, or a factor which interacts therewith (or a portion thereof), either naturally occurring or recombinant in origin, is contacted with a test compound and the ability of the test compound to bind to, or otherwise modulate the biological activity of, the eIF-4E or biologically active portion thereof is determined. Preferred biologically active portions of the eIF-4E to be used in assays of the present invention include fragments which participate in interactions with other translation initiation factors, or parts thereof, with the cap structure, with enzymes or factors which modulate the activity of eIF-4E or the activity of factors interacting therewith, or fragments with high surface probability scores for protein-protein or protein-substrate interactions. Binding of the test compound to eIF-4E, or eIF-4E-interacting factors can be determined either directly or indirectly as described above. In a preferred embodiment, the assay includes contacting eIF-4E or a biologically active portion thereof with a known compound which binds eIF-4E to form an assay mixture, contacting the assay mixture with a test compound and determining the ability of the test compound to interact with eIF-4E, or a biologically active portion thereof, wherein determining the ability of the test compound to interact with an eIF-4E comprises determining the ability of the test compound to preferentially bind to eIF-4E or biologically active portion thereof as compared to the known compound.

In another embodiment, the assay is a cell-free assay in which eIF-4E or biologically active portion thereof is contacted with a test compound and the ability of the test compound to modulate (e.g., stimulate or inhibit) the activity of eIF-4E or biologically active portion thereof is determined. Determining the ability of the test compound to modulate the activity of eIF-4E can be accomplished, for example, by determining the ability of eIF-4E to bind to an eIF-4E target molecule by one of the methods described above for determining direct binding. Determining the ability of eIF-4E to bind to an eIF-4E target molecule can also be accomplished using a technology such as real-time Biomolecular Interaction Analysis (BIA, Sjolander, S. and Urbaniczky, C. (1991) Anal. Chem. 63:2338-2345 and Szabo et al. (1995) Curr. Opin. Struct. Biol. 5:699-705). As used herein, “BIA” refers to a technology for studying biospecific interactions in real time, without labeling any of the interactants (e.g. BIA core). Changes in the optical phenomenon of surface plasmon resonance (SPR) can be used as an indication of real-time reactions between biological molecules.

In an alternative embodiment, determining the ability of the test compound to modulate the activity of eIF-4E can be accomplished by determining the ability of the test compound to modulate the activity of an upstream or downstream effector of an eIF-4E target molecule. For example, the activity of the test compound on the effector molecule can be determined, or the binding of the effector to eIF-4E can be determined as previously described.

In more than one embodiment of the above assay methods of the present invention, it may be desirable to immobilize eIF-4E, a portion thereof, or an eIF-4E target molecule, to facilitate the separation of complexed from uncomplexed forms of one or both of the proteins, as well as to accommodate automation of the assay. Binding of a test compound to eIF-4E or interaction of eIF-4E with a target molecule in the presence and absence of a candidate compound, can be accomplished in any vessel suitable for containing the reactants. Examples of such vessels include microtitre plates, test tubes and micro-centrifuge tubes. In one embodiment a fusion protein can be provided which adds a domain that allows one or both of the proteins to be bound to a matrix. For example, glutathione-S-transferase/eIF-4E fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or eIF-4E and the mixture incubated under conditions conducive to complex formation (e.g. at physiological conditions for salt and pH). Following incubation the beads or microtitre plate wells are washed to remove any unbound components, the matrix immobilized in the case of beads, complex determined either directly or indirectly, for example, as described above. Alternatively, the complexes can be dissociated from the matrix, and the level of eIF-4E binding or activity determined using standard techniques.

Other techniques for immobilizing proteins on matrices (and well-known in the art) can also be used in the screening assays of the invention. For example, either eIF-4E or an eIF-4E target molecule can be immobilized utilizing conjugation of biotin and streptavidin. Biotinylated eIF-4E or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with eIF-4E or target molecules but which do not interfere with binding of eIF-4E to its target molecule can be derivatized to the wells of the plate, and unbound target or eIF-4E trapped in the wells by antibody conjugation. Methods for detecting such complexes, in addition to those described above for the GST-immobilized complexes, include immunodetection of complexes using antibodies reactive with the eIF-4E or target molecule, as well as enzyme-linked assays which rely on detecting an enzymatic activity associated with eIF-4E or a target molecule thereof.

In another preferred embodiment candidate, or test compounds or agents are tested for their ability to inhibit or stimulate or regulate the phosphorylation state of eIF-4E, or portion thereof, or an upstream or downstream target protein, using for example an in vitro kinase assay. Kinase assays using eIf-4E and target molecules have been described herein and are well known in the art. Briefly, eIF-4E can be incubated with radioactive ATP , e.g., [gamma-³²P]-ATP , in a buffer containing MgCl₂ and MnCl₂, e.g., 10 mM MgCl₂ and 5 mM MnCl₂. Following the incubation, the immunoprecipitated eIF-4E can be separated by SDS-polyacrylamide gel electrophoresis under reducing conditions, transferred to a membrane, e.g., a PVDF membrane, and autoradiographed. The appearance of detectable bands on the auto radiograph indicates that eIF-4E has been phosphorylated. Phosphoaminoacid analysis of the phosphorylated substrate can also be performed in order to determine which residues on the eIF-4E are phosphorylated. Briefly, the radiophosphorylated protein band can be excised from the SDS gel and subjected to partial acid hydrolysis. The products can then be separated by one-dimensional electrophoresis and analyzed on, for example, a phosphoimager and compared to ninhydrin-stained phosphoaminoacid standards. Assays such as those described in, for example, Frederickson R. et al. (1992).

In yet another preferred embodiment candidate or test compounds or agents are tested for their ability to inhibit or stimulate eIF-4E-dependent modulation of cellular proliferation, using for example, the assays described herein (also see, 17 and 21).

In another preferred embodiment, candidate or test compounds or agents are tested for their ability to inhibit or stimulate cap dependent translation of mRNAs. For example, modulators of eIF-4E level and/or activity (direct or indirect) are identified in a method wherein a cell, or extract thereof is contacted with at least one candidate compound and the expression of a translation product whose translation is cap-dependent (or transcription product whose level is dependent on the level of a particular translation product whose expression is cap-dependent) is determined. The level of the translation product is determined in the presence of the candidate compound and compared to the level of expression thereof in the absence of the candidate compound. The candidate compound can then be identified as a modulator of eIF-4E level and/or activity based on this comparison. For example, when expression of a cap-dependent translation product is greater (statistically significantly greater) in the presence of the candidate compound than in its absence, the candidate compound is identified as a stimulator of eIF-4E level and/or activity (eIF-4E is a limiting translation initiation factor (15)). Alternatively, when expression of a cap-dependent translation product is lower (statistically significantly lower) in the presence of the candidate compound than in its absence, the candidate compound is identified as an inhibitor of eIF-4E level and/or activity. The level of eIF-4E protein, mRNA or post-translationally modified eIF-4E in the cells (or extracts thereof) can be determined by methods described herein or other methods known in the art for detecting eIF-4E mRNA, protein or post-translational modifications thereof.

The assays described above may be used as initial or primary screens to detect promising lead compounds for further development. Often, lead compounds will be further assessed in additional, different screens. Therefore, this invention also includes secondary eIF-4E (and eIF-4E-modulating agent) screens which may involve a number of assays utilizing mammalian cell lines expressing eIF-4E, parts thereof, eIF-4E-interacting proteins, and the like.

Tertiary screens may involve, for example, the study of the identified modulators in rat and mouse cancer models (conventional or transgenic models). Accordingly, it is within the scope of this invention to further use an agent identified as described herein in an appropriate animal model. For example, a test compound identified as described herein (e.g., an eIF-4E modulating agent, an antisense eIF-4E nucleic acid molecule, an eIF-4E-specific antibody, or an eIF-4E-binding partner) can be used in an animal model to determine the efficacy, toxicity, or side effects of treatment with such an agent. In addition, an agent identified as described herein can be used in an animal model to determine the mechanism of action of such an agent. Furthermore, this invention pertains to uses of novel agents identified by the above-described screening assays for treatment (e.g. treatment of different types of diseases associated with aberrantly modulated apoptotic pathways), as described herein. Of note, one such compound or agent, 4E-BP1, has been shown to induce apoptose in transformed cells but to lack toxicity or detectable undesirable effects in non-transformed cells.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91:11422; Zuckermann et al. (1994), J. Med. Chem. 37:2678; Cho et al. (1993) Science 261 :1303; Carrell et al. (1994) Angew. Chem, Int. Ed Engl. 33:2059; Carell et al. (1994) Angew. Chem. Jnl. Ed. Engl. 33:2061; and in Gallop et al. (1994). Med Chem. 37:1233. Libraries of compounds may be presented in solution (e.g. Houghten (1992) Biotechniques 13:412-421). or on beads (Lam (199]) Nature 354:82-84), chips (Fodor (1993) Nature 364:555-556). bacteria (Ladner U.S. Pat. No. 5.223,409), spores (Ladner U.S. Pat. No. '409), plasmids (Cull et al.(1992) Proc Natl Acad Sci USA 89:1865-1869) or on phage (Scott and Smith (1990); Science 249:386-390). Examples of methods for the synthesis of molecular libraries can be found in the art, for example in: DeWitt et al. (1993) Proc. Natl. Acad. Sci. USA. 90:6909; Erb et al. (1994) Proc. Natl. Acad Sci. USA 91: 11422; Zuckermann et al. (1994), .J: Med. Chem. 37:2678; Cho et al. (1993), Science 261 :1303; Carrel1 et al. (1994) Angew. Chem Int. Ed. Engl. 33:2059, or luciferase, and the enzymatic label detected by determination of conversion of an appropriate substrate to product.

In summary, based on the disclosure herein, those skilled in the art can develop eIF-4E screening assays which are useful for identifying compounds for use in disorders displaying an aberrantly modulated apoptosis pathway. The assays of this invention may be developed for low-throughput, high-throughput, or ultra-high throughput screening formats.

The assays of this invention employ either natural or recombinant eIF-4E. Cell fraction or cell-free screening assays for modulators of eIF-4E biological activity can use in situ, purified, or purified recombinant eIF-4E. Cell based assays can employ cells which express eIF-4E naturally, or which contain recombinant eIF-4E gene constructs, which constructs may optionally include inducible promoter sequences. In all cases, the biological activity of eIF-4E can be directly or indirectly measured; thus modulators of eIF-4E biological activity can be identified. The modulators themselves may be further modified by standard combinatorial chemistry techniques to provide improved analogs of the originally identified compounds.

In one embodiment, at least one of the 4E-BP1 and eIF4E interacting domains of the present invention may be provided as a fusion protein. The design of constructs therefor and the expression and production of fusion proteins are well known in the art (Sambrook et al., 1989, supra; and Ausubel et al., 1994, supra). In a particular embodiment, both interaction domains are part of fusion proteins. A non-limiting example of such fusion proteins includes a LexA-4E-BP1 fusion (DNA-binding domain-4E-BP1; bait) and a B42-eIF4E fusion (transactivator domain-eIF4E; prey). In yet another particular embodiment, the LexA-4E-BP1 and B42-eIF4E fusion proteins are expressed in a yeast cell also harboring a reporter gene operably linked to a LexA operator and/or LexA responsive element. Of course, it will be recognized that other fusion proteins can be used in such 2 hybrid systems. Furthermore, it will be recognized that the fusion proteins need not contain the full-length 4E-BP1 or eIF4E polypeptide. Indeed, fragments of these polypeptides, provided that they comprise the interacting domains, can be used in accordance with the present invention.

Non-limiting examples of such fusion proteins include a hemaglutinin fusions, Gluthione-S-transferase (GST) fusions and Maltose binding protein (MBP) fusions. In certain embodiments, it might be beneficial to introduce a protease cleavage site between the two polypeptide sequences which have been fused. Such protease cleavage sites between two heterologously fused polypeptides are well known in the art.

For certainty, the sequences and polypeptides useful to practice the invention include without being limited thereto mutants, homologs, subtypes, alleles and the like. It shall be understood that generally, the sequences of the present invention should encode a functional (albeit defective) interaction domain. It will be clear to the person of ordinary skill that whether an interaction domain of the present invention, variant, derivative, or fragment thereof retains its function in binding to its partner can be readily determined by using the teachings and assays of the present invention and the general teachings of the art.

In certain embodiments, it might also be beneficial to fuse the interaction domains of the present invention to signal peptide sequences enabling a secretion of the fusion protein from the host cell. Signal peptides from diverse organisms are well known in the art. Bacterial OmpA and yeast Suc2 are two non limiting examples of proteins containing signal sequences. In certain embodiments, it might also be beneficial to introduce a linker (commonly known) between the interaction domain and the heterologous polypeptide portion. Such fusion protein find utility in the assays of the present invention as well as for purification purposes, detection purposes and the like.

As exemplified herein below, the interaction domains of the present invention can be modified, for example by in vitro mutagenesis, to dissect the structure-function relationship thereof and permit a better design and identification of modulating compounds. However, some derivative or analogs having lost their biological function of interacting with their respective interaction partner (e.g. 4E-BP1 or eIF4E) may still find utility, for example for raising antibodies. Such analogs or derivatives could be used for example to raise antibodies to the interaction domains of the present invention. These antibodies could be used for detection or purification purposes. In addition, these antibodies could also act as competitive or non-competitive inhibitor and be found to modulate eIF4E-dependent translation. Some of these antibodies might even be used as eIF4E sequestering agents ‘per se’.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra). The use of a mammalian cell as indicator can provide the advantage of furnishing an intermediate factor, which permits for example the interaction of two polypeptides which are tested, that might not be present in lower eukaryotes or prokaryotes. Of course, this advantage might be rendered moot if both polypeptide tested directly interact. It will be understood that extracts from mammalian cells for example could be used in certain embodiments, to compensate for the lack of certain factors. It will be realized that the field of translation provides ample teachings of methods to prepare and reconstitute translation extracts.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody—A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

From the specification and appended claims, the term therapeutic agent should be taken in a broad sense so as to also include a combination of at least two such therapeutic agents. Further, the DNA segments or proteins according to the present invention can be introduced into individuals in a number of ways. For example, erythropoietic cells can be isolated from the afflicted individual, transformed with a DNA construct according to the invention and reintroduced to the afflicted individual in a number of ways, including intravenous injection. Alternatively, the DNA construct can be administered directly to the afflicted individual, for example, by injection in the bone marrow. The DNA construct can also be delivered through a vehicle such as a liposome, which can be designed to be targeted to a specific cell type, and engineered to be administered through different routes.

In one particular embodiment, the present invention provides the means to reverse a Ras-induced chemoresistance of cancer cells which comprises an increase in the level of expression of an eIF4E sequestering agent, or an increase in its eIF4E-sequestering activity. In one particular embodiment, the 4E-BP1 level of expression or its activity in sequestering eIF4E is increased. It will be recognized that having shown that an upregulation of 4E-BP1 (level or activity) selectively activates apoptosis in Ras-transformed cells, provides numerous means of achieving the triggering of apoptosis in Ras-transformed cells. Non-limiting examples of such means include 4E-BP1 mutants, mutants of the eIF4E interaction domain of 4E-BP1, eIF4E ligands (e.g. antibodies), eIF4E antisense, and the like. Broadly, the present invention provides methods to reverse Ras-induced chemoresistance of cancer cells (or alternatively the triggering of the pro-apoptotic pathway) by decreasing the level and/or efficiency of eIF4E-dependent translation (or alternatively by increasing the level and/or efficiency of eIF-4E dependent translation).

It should also be understood that although the present invention relates in particular to a proapoptotic pathway induction in cancer cells, that the present invention provides the means to modulate translation and/or induce apoptosis in cells displaying a rapid growth rate and/or abnormal proliferation. Non-limiting examples thereof include cells associated with proliferative diseases, inflammation, psoriasis and the like.

For administration to humans, the prescribing medical professional will ultimately determine the appropriate form and dosage for a given patient, and this can be expected to vary according to the chosen therapeutic regimen (e.g. DNA construct, protein, molecule), the response and condition of the patient as well as the severity of the disease.

Composition within the scope of the present invention should contain the active agent (e.g. protein, nucleic acid, or molecule) in an amount effective to achieve the desired therapeutic effect while avoiding adverse side effects. Typically, the nucleic acids in accordance with the present invention can be administered to mammals (e.g. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th Ed., Mack Ed.). For the administration of polypeptides, antagonists, agonists and the like, the amount administered should be chosen so as to avoid adverse side effects. The dosage will be adapted by the clinician in accordance with conventional factors such as the extent of the disease and different parameters from the patient. Typically, 0.001 to 50 mg/kg/day will be administered to the mammal.

### BRIEF DESCRIPTION OF THE DRAWINGS

Having thus generally described the invention, reference will now be made to the accompanying drawings, showing by way of illustration a preferred embodiment thereof, and in which:

FIG. 1 shows that rapamycin but not cycloheximide abrogates Ras-induced resistance to apoptosis. (A) Western blot analysis of Ras expression patterns in exponentially proliferating CREF transfected with an empty vector (neo) or with vector encoding RasV12 (12). Cell lysates (50 μg cellular protein/lane) were subjected to 14% SDS-polyacrylamide gel electrophoresis (PAGE), and transferred to nitrocellulose. Immunoblot analysis was performed with anti-Ras (1:500, Transduction Laboratories) or anti-actin (loading control, 1:100, Sigma) antibody. Densitometric quantitation (O.D.) is displayed in arbitrary units. (B) Effect of rapamycin on cell viability. CREF/Neo (−) and CREF/RasV12 (+) cells were cultured with (+) or without (−) 75 nM rapamycin for 24 h in regular growth medium (solid columns) or in growth medium supplemented either with 5 μM lovastatin (striped columns) or 75 nM camptothecin (shaded columns). Cells were fixed, stained with propidium iodide, and apoptosis was quantified by flow cytometry, as described (18). Values shown represent the mean±SD (n=3) of the percentage of cells with sub-diploid DNA content. (C) Apoptosis in the presence of translational inhibitors. CREF/RasV12 were cultured for 4 h in growth medium containing different concentrations of either rapamycin (closed circles) or cycloheximide (open circles). To quantify the rate of protein synthesis, growth medium was replaced with methionine-free medium containing 10% dialyzed serum and [³⁵S] methionine (15 μCi/ml; SA=6,000 Ci/mMole) for 3 h and TCA precipitable radioactivity was quantified by liquid scintillation counting. Parallel cultures were continued for 24 h in growth medium with or without 5 μM lovastatin in the continued presence of the indicated translational inhibitors. Apoptosis is expressed as an increase in the percentage of that observed in the presence of lovastatin alone (range 2 to 8%).

FIG. 2 shows that ectopic 4E-BP1 selectively activates apoptosis in Ras-transformed cells. CREF/Ras (A, B, and C) and CREF (D, E, and F) were stably transfected with either an empty expression vector (puro) or vector encoding full length wild type human 4E-BP1 (23), and puromycin resistant clones were isolated. (A and D). Cells were lysed using three freeze-thaw cycles and 40 μg of boiled extracts were analyzed by Western blotting with rabbit anti-4E-BP1 (1:2500) (8) followed by peroxidase conjugated anti-rabbit IgG (1:4000, Sigma). (B and E). Micrographs (×300) of Ras-transformed (B) and non-transformed CREF (E) ectopically expressing wild type 4E-BP1 (BP1wt) or an empty puro vector (puro). CREF/Ras/BP1wt (clone 14), CREF/BP1wt (clone 4), and mock transfected cells were plated one week after clonal isolation, cultivated for 24 h in growth medium, and stained with acridine orange after fixation with 70% ethanol. (C and F). To quantify apoptosis, cells were cultured for 24 h in growth medium without cytostatic drugs (solid columns) or with either 5 μM lovastatin (striped columns) or 75 nM camptothecin (shaded columns). Data are normalized to the apoptotic frequency in cells transfected with an empty puro vector (3.2±1.1%). Each point represents the mean±SD (n=3).

FIG. 3 shows that 4E-BP1-promoted apoptosis in Ras-transformed cells is associated with displacement of the translation factor eIF4GI from eIF4E. (A) Immunoblot analysis of 4E-BP1 and eIF4GI associated with cap-bound eIF4E in clones of CREF/RasV12 ectopically expressing 4E-BP1. Lysates from each clone (250 μg) were incubated with m⁷GTP-Sepharose resin (Amersham Pharmacia Biotech) to capture eIF4E and its binding partners (8). Samples were eluted with a buffer containing 70 μM m⁷GTP. Cap-bound material was subjected to SDS-PAGE, and transferred to nitrocellulose. Blots were probed first for eIF4E (mouse monoclonal antibody, 1:500, Transduction Laboratories), then stripped and probed for 4E-BP1 (rabbit polyclonal antiserum, 1:2500, Transduction Laboratories) (8), then stripped and probed a third time for eIF4GI (rabbit polyclonal antibody, 1:4000). Apoptosis is shown as a function of the 4E-BP1/eIF4E (B) or eIF4G/eIF4E (C) ratio in clones incubated in growth medium for 24 h in the presence (open circles) or absence (closed circles) of 5 μM lovastatin.

FIG. 4 shows that 4E-BP1 lacking an eIF4E binding domain does not promote apoptosis. CREF/RasV12 were transiently transfected with either a pACTAG-2 construct encoding hemagglutinin (HA) tagged human wild type 4E-BP1 (4E-BP1wt), a pACTAG-2 vector encoding HA tagged 4E-BP1 with an internal deletion of amino acids 51-67 (4E-BP1Δ) which includes the eIF4E binding domain (amino acids 51-60) (24), or an empty vector (HA vector). Transfection (1 μg plasmid DNA, 24 h) was conducted using the FuGENE™ transfection reagent (Boehringer Mannheim) in accord with the protocol provided by the manufacturer. Transfected and non-transfected CREF/RasV12 were incubated in growth medium with or without 7.5 μM lovastatin for 48 h and fixed with absolute methanol. To detect the level of HA expression, cells were incubated for 16 h at 4° C. with mouse anti-HA IfG_{2bk} antibody (4 μg/ml, Boehringer Mannheim) or with mouse isotype specific IgG_{2bk} antibody (4μg/ml, PharMingen), followed by incubation with fluorescein-conjugated anti-mouse IgG antibody (1:40, Sigma) for 30 min. All samples were also stained with propidium iodide for 20 min prior to measurement of non-specific (open histograms) or HA-specific (closed histograms) green emission of fluorescein, and red emission of propidium stained DNA (shaded histograms) using standard optics of the FACScan flow cytometer (Becton Dickinson) and the CellQuest program. The results of a representative experiment are shown (three independent transfection experiments yielded similar results).

FIG. 5 shows the sequence alignment of the 4E-binding site of 4E-BPs, as well as the consensus sequence which could be used as a 4E sequestering agent or for the development of further 4E sequestering agents. The light gray indicates positions at which mutation to alanine abrogates the binding to eIF4E (Mader et al., 1995; and Poulin et al., 1998). The dark gray indicates highly conserved amino acid positions. +/− indicate charged amino acids. φ refers to hydrophobic amino acids, Y refers to tyrosine, f refers to phenylalanine, although an absolute requirement for this amino acid does not appear to be necessary based on the dyctostelium discoideum consensus sequence. L refers to leucine, and “.” shows that the 4E binding site at this particular position is not dependent on a particular amino acid. h=human (mouse/rat have the same sequence in his region); gg=gallus gallus (chicken); hr=halocynthia roretzi; bm=bombyx mori; sm=schistosoma mansoni; dd=dictyostelium discoideum. Y, L, R, S, and P refer to the standard one letter code for amino acids.

FIG. 6 shows the alignment of 4E-binding sites comprised in a number of diverse eIF4E-binding proteins. The light gray indicates positions at which a mutation to alanine abrogates the binding to eIF4E (Mader et al., 1995; and Poulin et al., 1998). +/− indicate charged amino acids. φ refers to hydrophobic amino acids. Y and L refer to the standard one letter code for amino acids.

FIG. 7 shows the selective activation of the eIF4F translation complex in breast cancer cells. (a) Western blot of cellular eIF4E, eIF4G1, and 4E-BP1. (b) Immunoblot analysis of eIF4GI associated with cap-bound eIF4E. For the cap-affinity assay, cell lysates (250 ug) were incubated with 7-methyl-GTP Sepharose resin (Amersham Pharmacia Biotech) to capture eIF4E and its binding partners. Samples were eluted with buffer containing 70 □M 7-methyl-GTP. Cap bound material was subjected to SDS PAGE and transferred to nitrocellulose. Blots were probed for eIF4E (mouse monoclonal antibody, 1:500, Transduction Laboratories), and for eIF4GI (rabbit polyclonal antibody, 1:4000). (c) Cells were incubated in the presence of 7.5 □M lovastatin or 500 nM camptothecin for 24 h, and percentages of cell with hypodiploid DNA contents (% of apoptosis) were quantified by flow cytometry Each bar represents the mean±SD (4 independent replications).

FIG. 8 shows that 4E-BP1 displaces eIF4G1 from eIF4E and sensitizes MDA-MB-231 breast cancer cells to apoptosis. (a,b)Immunoblot analysis of HA4E-BP1 expression (a) and binding of eIF4G1 to the cap-captured eIF4E (b), in three clonal cell lines of MDA-MB-231 transfected with a construct encoding HA-4E-BP1. (c) Summative analysis of apoptotic frequencies in twelve MDA-MB-231 clonal cell lines ectopically expressing 4E-BP1. Cells were cultured for 24 h+/−7.5 □M lovastatin or 200 nM camptothecin. Apoptosis was quantified by flow cytometry. Each bar represents the mean±SD (3 independent replications).

FIG. 9 shows the morphological hallmarks of apoptosis and caspase-3 activity in MDA-MB-231 breast cancer cells ectopically expressing wild type 4E-BP1 (BP1-wt). Cells were incubated in the presence or absence of 7.5 uM lovastatin for 24h and immunostaining for active caspase-3 and staining of nuclei with DAPI was carried out. Arrows highlight apoptotic cells and demonstrate that cells with apoptotic nuclei are caspase-3 positive, consistent with the order of events during apoptosis.

FIG. 10 shows that the 4E-BP1-promoted drug susceptibility in lung cancer cells is associated with displacement of translation factor eIF4G1 from the eIF/4E/cap complex. NSCLC cells (line 2009) were transfected with HA-tagged 4E-BP1 and both mock (neo) and 4E-BP1 (BP1) transfected cells were subjected to immunoblotting and apoptosis assays as described in the legend to FIGS. 4 and 5. (a) Western blot of cellular (the fast migrating form) and HA-tagged ectopic (the slow migrating form) 4E-BP1. (b) Immunoblot assay of eIF4G1 and 4E-BP1 associated with cap-bound eIF4E. (c) Flow cytometric analysis of apoptosis (shown are results of one pilot experiment).

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

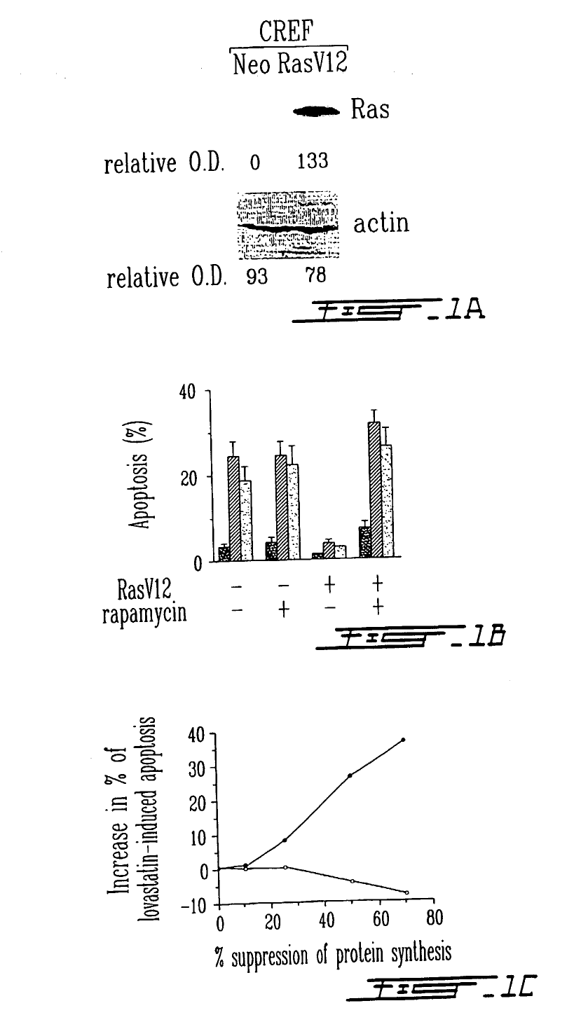

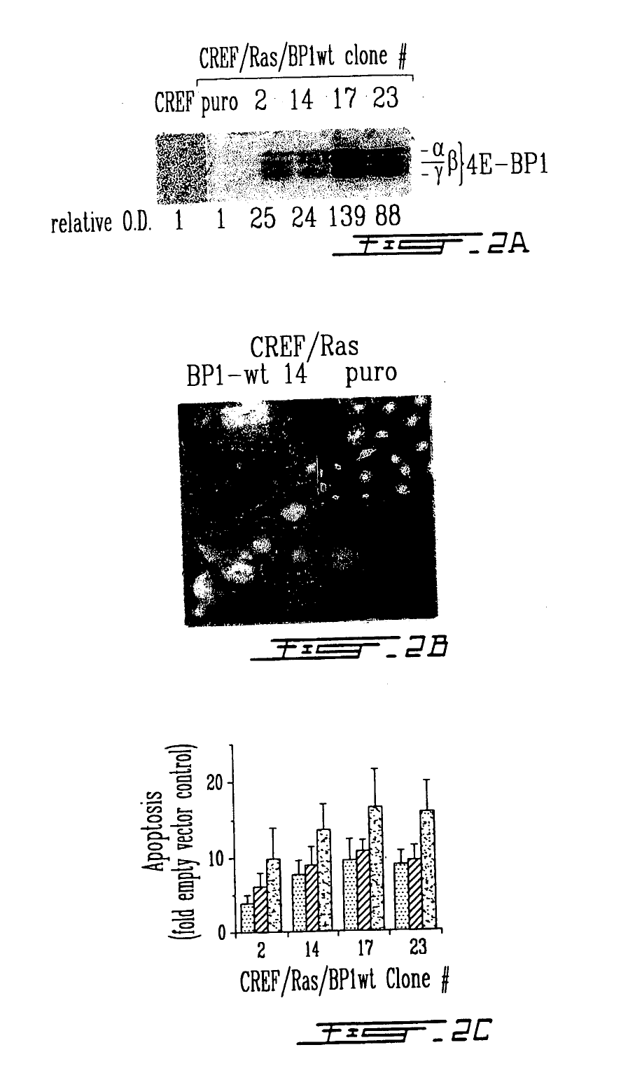

Herein, the inventors sought to identify the molecular target of FRAP kinase conferring Ras-induced viability and chemoresistance. To that end, a cell system (12) was used, in which constitutively expressed oncogenic RasV12 enables cloned rat embryo fibroblasts (CREF) to survive in otherwise lethal concentrations of cytostatic drugs (non genotoxic, lovastatin; genotoxic, camptothecin, FIG. 1). Rapamycin completely abrogated Ras-dependent resistance to drug-induced cell death (FIG. 1B); and even when applied as a single agent, stimulated apoptosis in cells expressing activated Ras. This pro-apoptotic effect of rapamycin was not observed in non-transformed fibroblasts. This observation confirms a dual pro-apoptotic and anti-apoptotic function for RasV12, and implicates FRAP in Ras-dependent rescue from both Ras activated and drug-triggered apoptotic pathways.

In accord with previous reports (9), rapamycin also caused a dose-dependent decline in protein synthesis which paralleled its ability to sensitize Ras-transformed cells to lovastatin-induced apoptosis (FIG. 1C). Of note, equipotent doses of the peptide elongation inhibitor cycloheximide actually blocked apoptosis. This is consistent with recent findings demonstrating that the execution of lovastatin-induced cell death requires global protein synthesis (13), and suggests that a generalized inhibition of mRNA translation is not the means by which rapamycin exerts its pro-apoptotic effect.

An alternative mechanism is that rapamycin predominantly inhibits translation of a specific set of mRNA required for Ras survival signaling and chemoresistance. In support of this idea, Ras function has been closely linked to the initiation of cap-dependent protein synthesis. Cell transformation by oncogenic Ras requires increased activity of translation initiation factor eIF4E (14), the mRNA cap binding protein which functions during translation of cellular mRNAs possessing the 5′ cap structure (15). The cap is bound by the initiation complex eIF4F, which in mammalian cells consists of the bi-directional RNA helicase eIF4A, the docking protein eIF4G, and the cap binding subunit eIF4E. eIF4E is considered to be rate-limiting for translation initiation under most circumstances, and a major target for regulation (16).

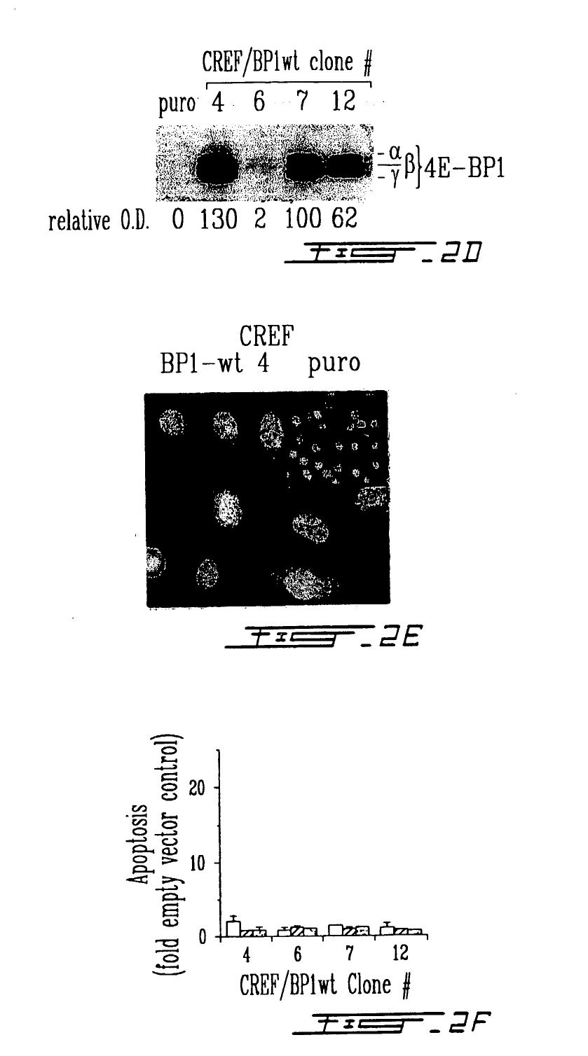

To explore whether the 4E-BPs modulate Ras-dependent viability and chemoresistance, CREF/RasV12 and CREF were engineered to constitutively express wild type 4E-BP1 (BP2wt) linked to a puro selectable marker, and puromycin resistant clones were isolated (see Example 2). All mock-transfected, puromycin resistant clones (n=6) were stable. Each displayed viability and biochemical characteristics similar to those of parental CREF/RasV12. Four viable CREF/Ras/BPwt clonal lines were developed and assayed for steady state levels of 4E-BP1. Under conditions in which expression of endogenous 4E-BP1 in all mock-transfected cells was undetectable (70 μg cellular protein/lane; 15 sec. development time), CREF/Ras/BP1wt clones displayed a range of ectopic 4E-BP1 expression. Western blot analysis performed on total cellular extracts (FIG. 2A) revealed human 4E-BP1 represented by hypo-, intermediate-, and hyperphosphorylated forms (19). Many cells comprising the CREF/RasV12/BP1wt clonal lines displayed morphological hallmarks of apoptosis, such as cell shrinkage, chromatin condensation and fragmentation of nuclei (FIG. 2B). Quantification of apoptosis by flow cytometry revealed a 2 to 8-fold increase in basal apoptosis in complete medium, suggesting that ectopic 4E-BP1 shifted Ras signaling from suppression to induction of apoptosis (FIG. 2C). Cytostatic drugs approximately doubled the basal apoptotic frequency in all clones, indicating that Ras-dependent chemoresistance was lost in cells ectopically expressing 4E-BP1.

In contrast to results with transformed CREF/RasV12, overexpressed 4E-BP1 did not activate apoptosis in non-transformed parental CREF lacking activated Ras. Four clonal lines representing a wide spectrum of 4E-BP1 expression were subjected to further analysis (FIG. 2D). Ectopic expression of 4E-BP1 did not alter the morphology of CREF (FIG. 2E), nor did it alter their viability (FIG. 2F). These findings indicate that physiologically regulated wild type Ras does not cooperate with 4E-BP1 in sensitizing fibroblasts to apoptosis, but rather that overexpressed oncogenic Ras is required.

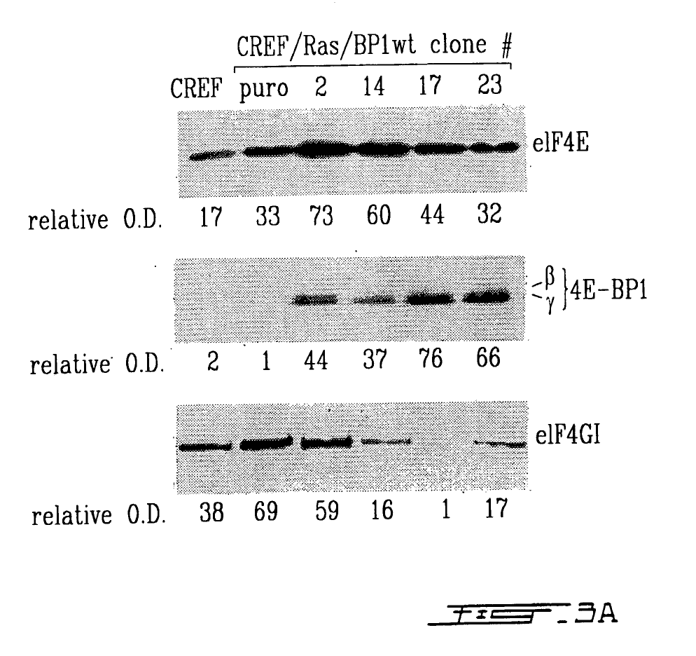

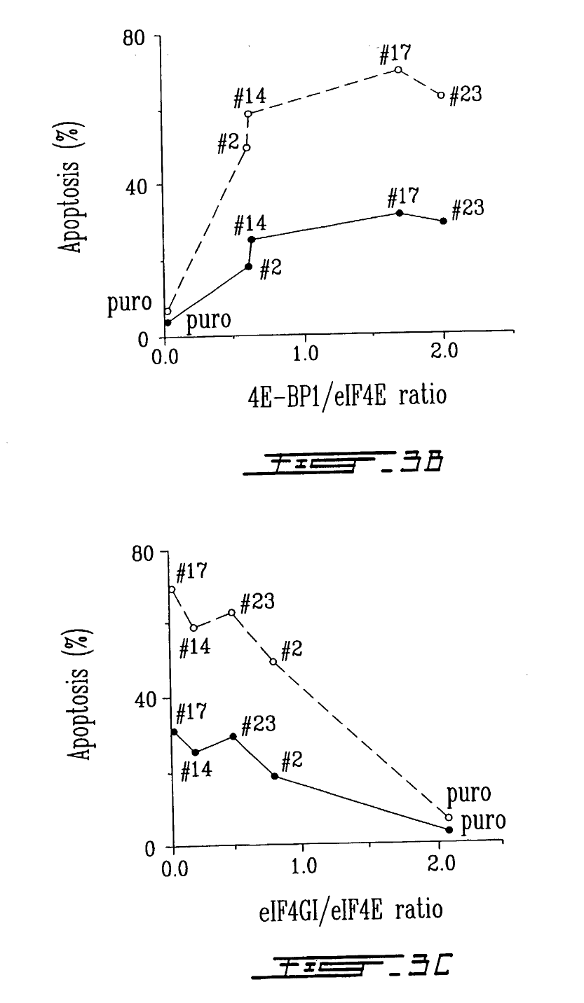

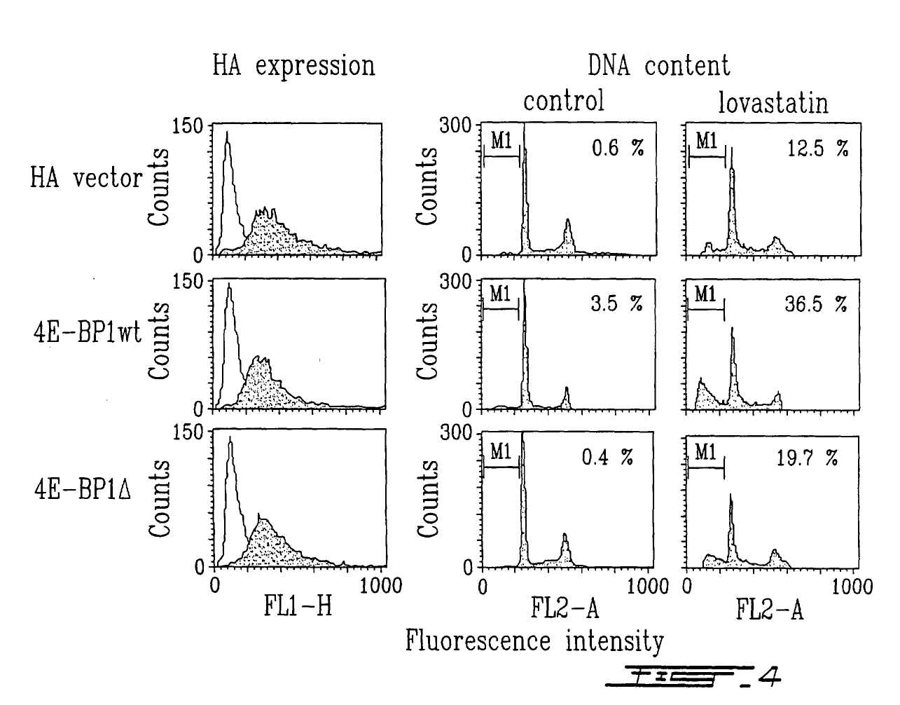

Next, it was investigated whether the pro-apoptotic function of 4E-BP1 in CREF/RasV12 was associated with its ability to sequester eIF4E from the translationally active eIF4E/eIF4GI complex. Cellular extracts were incubated with the cap-analog m⁷GTP-agarose to capture eIF4E and its cellular binding partners. The levels of cap-bound eIF4E, 4E-BP1, and eIF4GI were quantified by sequential immunoblotting and densitometry. Each CREF/RasV12/BP1wt clone displayed eIF4E associated with significantly increased amounts of fast migrating, hypophosphorylated 4E-BP1 (FIG. 3A). Consistent with this, clones ectopically expressing 4E-BP1wt also manifested decreased amounts of eIF4GI bound to eIF4E, confirming the ability of ectopic 4E-BP1 to inhibit assembly of the eIF4F pre-initiation complex. The apoptotic frequency in clones co-expressing activated Ras and 4E-BP1 was proportional to the amount of 4E-BP1 complexed with eIF4E (FIG. 3A), and inversely related to the eIF4GI/eIF4E ratio (FIG. 3C). Thus, the ability of 4E-BP1 to stimulate apoptotic death was a function of its activity in competitively displacing eIF4GI from eIF4E.

To determine whether the interaction of 4E-BP1 with eIF4E was necessary for the pro-apoptotic function of 4E-BP1 in Ras-transformed cells, a 4E-BP1 deletion mutant (4E-BP1Δ) which lacks the eIF4E binding site (24) was used. Transient transfection of CREF/RasV12 with 4E-BP1wt enhanced spontaneous apoptosis and chemosensitivity to lovastatin in a manner similar to that observed in the stable CREF/RasV12/BP1wt clones. In marked contrast, transient transfection with 4E-BP1Δ had minimal effects on viability, despite similar levels of gene expression. Thus, the ability of 4E-BP1 to bind eIF4E was essential for its blockade of Ras-induced survival signaling.

The finding that 4E-BP1, eIF4E and eIF4G are implicated in the apoptotic pathway emanating from oncogenic Ras through FRAP is of pharmacological value, as specific modulation of the 4E-BP1-eIF4E interaction, as well as the modulation of the formation of the eIF4F preinitiation complex and of the level of eIF4E complex to eIF4G1, could be used to modulate this apoptotic pathway. This possibility is particularly intriguing in light of the fact that overexpression of 4E-BP1 did not activate apoptosis in non-transformed cells lacking activated Ras.

Furthermore, the present invention, having identified translation initiation through eIF4E and its association with eIF4G as a biochemical pathway involved in modulation of apoptosis, provides numerous assays and methods to screen and identify such apoptosis modulators and especially pro-apoptotic agents.

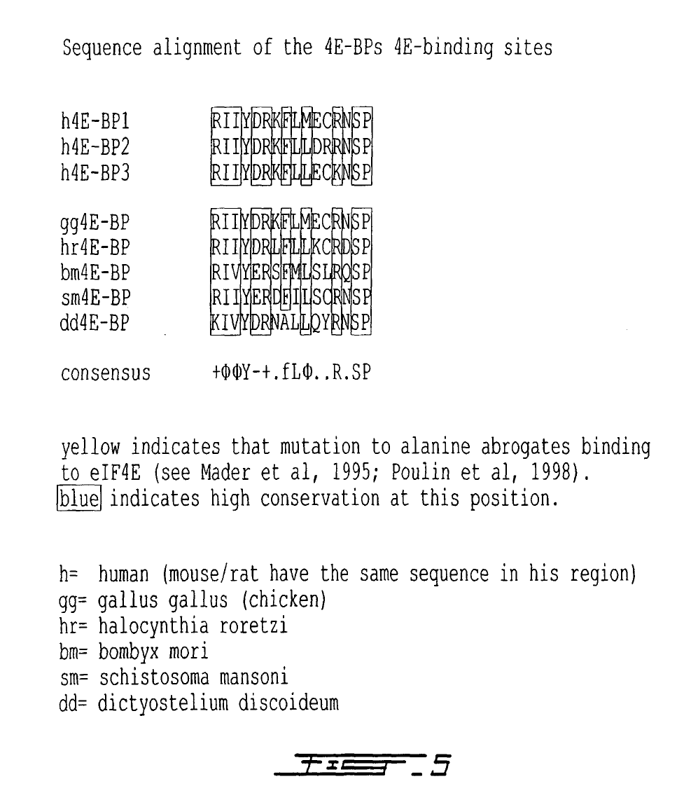

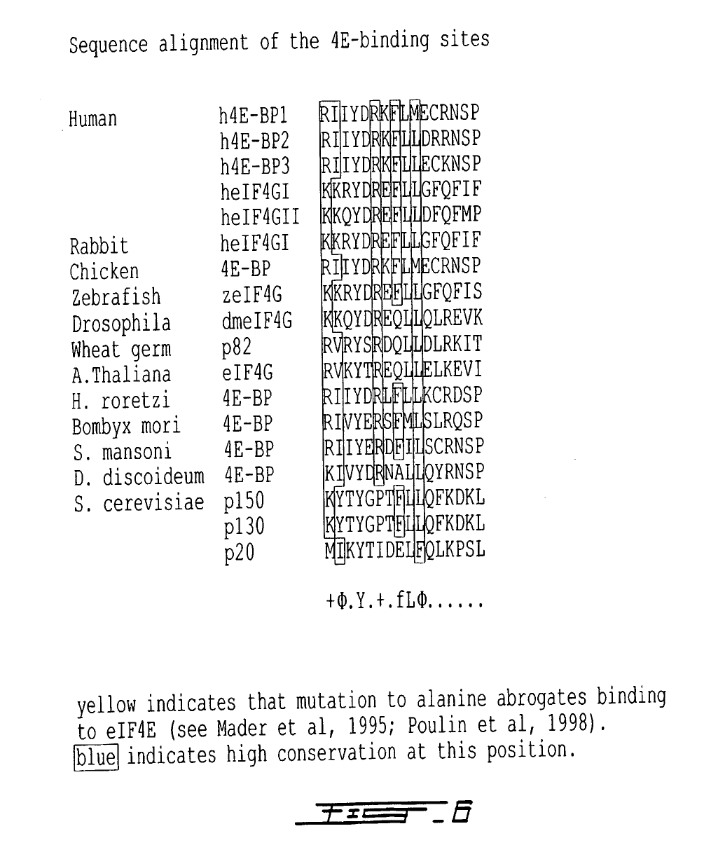

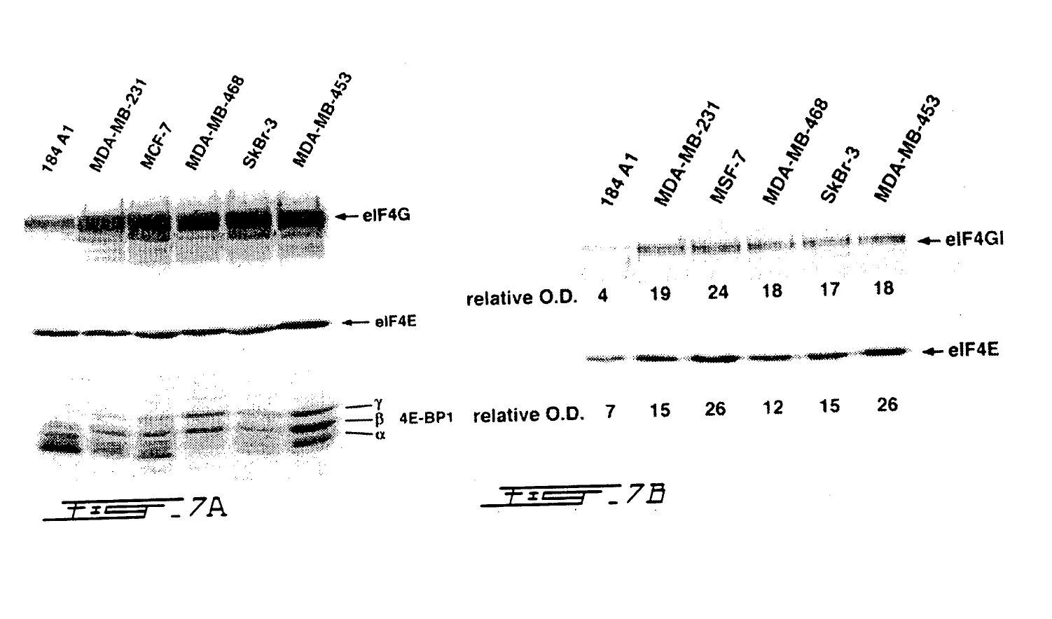

As seen in FIGS. 5 and 6, the eIF4E binding sites (or eIF4E interaction domains) of numerous protein from evolutionarily distant organisms show a significant homology/identity. In addition, the sequences of rat and mouse 4E-BP1, 4E-BP2 and 4E-BP3 are 100% identical to those of the human in the region presented here.

Indeed, consensus sequences which retain their eIF4E binding activity are provided. These consensus sequences could be used as eIF4E sequestering agents or as starting points to design other eIF4E sequestering agents.

Of note, recombinant peptides derived from 4E-BP1 and eIF4GII have been shown to inhibit translation of mRNAs (Marcotrigiano et al., 1999, Molecul. Cell 3:707-716).

The present invention is illustrated in further detail by the following non-limiting examples.

### EXAMPLE 1

### Cells and Cell Culture Conditions

Parental cloned rat embryo fibroblasts (CREF) and their derivatives expressing empty vector (CREF/Neo) or H-Ras[V12] (CREF/RasV12) as well as CREF/RasV12 constitutively expressing eIF4E antisense mRNA (As4E cells) were kindly provided by Dr. A. De Benedetti (Louisiana State University, Shreveport, La.). Growth characteristics of CREF and CREF/RasV12 (the latter are also known as CREF T24) have been described (Boylan et al. 1990). Inhibitory effects of eIF4E antisense mRNA on eIF4E expression pattern, cell cycle transit, and tumorigenicity of CREF/RasV12 have been previously documented (Rinker-Schaeffer et al. 1993; Graff et al. 1995). All cells were maintained in Dulbecco's modified Eagle's medium supplemented with 10% FCS, 100 units/ml penicillin, 100 g/ml streptomycin and 250 ng/ml amphotericin B (Gibco).

### EXAMPLE 2

### Plasmids, Cell Transfection and Cloning

Plasmids encoding eIF4E antisense mRNA, the transfection procedure, and isolation of As4E clones have been described (De Benedetti et al. 1991; Rinker-Schaeffer et al. 1993; Graff etal. 1995). The coding sequence of human 4E-BP1 was amplified by PCR and directionally cloned into the EcoR1 and BamH1 sites of the mammalian expression vector pSRαpuro (a kind gift from Dr. P. Jolicoeur, Institut de Recherches Cliniques, Montreal, PQ, Canada). The insert was fully sequenced and no mutation were detected. Transfections of CREF and CREF/RasV12 were performed by calcium phosphate precipitation technique. Selective medium containing 4 g/ml puromycin was applied after 24 h and resistant clones were isolated after 12 to 16 days.

### EXAMPLE 3

### Apoptosis Assays and Cell Cycle Analysis

Apoptosis was induced by reducing serum concentration in the medium (0.1 to 0.5%) or by addition of cytostatic agents. Each cytostatic agent was used at a concentration that caused 75 to 80% of CREF to be reversibly arrested at the expected cycle locus (assessed by FACS, see below) as follows: 5 μM lovastatin, late G1; hydroxyurea, 1.5 mM, late G1/early S; camptothecin, 75 nM, G2; colcemide, 50 ng/mL, M phase. Frequency of apoptosis was quantified by flow cytometric analysis of the percentage of cells with hypodiploid DNA content, as described (Polunovsky et al. 1994; Polunovsky et al. 1996). Adherent and nonadherent cells were pooled, washed in phosphate-buffered saline (PBS), and fixed with ice cold ethanol (70%, 4° for 1 h). Fixed cells were washed with PBS and incubated in propidium iodide stain mixture (52 g/ml propidium iodide, 0.05% Triton X-100, 18 mg/ml EDTA, 100 U/ml ribonuclease in PBS). After incubation (45 min, 37°), DNA content was determined by quantitative flow cytometry (Becton-Dickinson FACS Star Plus). Results were tabulated as the mean±SD of 2 to 5 separate experiments. In each experiment, all conditions were examined in duplicate or triplicate. Flow cytometric data were confirmed by analysis of apoptosis after acridine orange or TUNEL staining (Polunovsky et al. 1994; Polunovsky et al. 1996). Cell cycle distribution was monitored by flow cytometric analysis of DNA content. Entry into S phase was quantified by analysis of BrdU incorporation into DNA, as described (Polunovsky et al. 1996).

### EXAMPLE4

### Metabolic Labeling and Protein Synthesis Assay

Exponentially proliferating cells (5×10⁴) were pretreated with potent translational inhibitors for 4 h in standard growth medium and transferred for 3 h into methionine-free medium containing 10% dialysed FCS and 15 Ci/ml [³⁵S] methionine (SA=6000 Ci/mMole). Cell monolayers were rinsed twice with PBS (4°), washed three times with 5% TCA (4°), and lysed with 0.1% NaOH containing 0.1% SDS. TCA precipitable radioactivity was quantified by liquid scintillation counting.

### EXAMPLE 5

### Immunoblot Analysis

For the Western blot assay, cells were collected by scraping into buffer A (150 mM NaCl, 50 mM Tris pH 7.5, 50 mM NaF, 1 mM EDTA, 1 mM EGTA, 1 mM DTT, 50 mM β-glycerophosphate, 10 mM Na pyrophosphate, 0.1 mM Na orthovanadate, 50 nM okadaic acid, 1 mM PMSF, 1 g/ml pepstatin A, 1 g/ml leupeptin), and cell membranes were disrupted by three successive freeze-thaw cycles. The cell extracts were microcentrifuged at 4° for 10 min and the supernatants were retained. For Ras, eIF4E and actin, supernatants were mixed with reducing sample buffer, boiled, and subjected to 14% PAGE, followed by electroblotting to nitrocellulose. For 4E-BP1, it was necessary to first boil the samples (7 minutes) to eliminate interfering proteins prior to electrophoresis. Western blotting was performed by blocking the membrane in Tris buffered saline, pH 7.5 containing 0.05% Tween 20 and 5% nonfat dry milk for 1 hour. After incubation with primary and peroxidase conjugated secondary antibodies, blots were developed by ECL (Amersham) and film images were scanned for densitometry using a Biorad GS700 Imaging Densitometer with Molecular Analyst software.

### EXAMPLE 6

### Quantitation of eIF4E and 4E-BP1

Cells were lysed as described above. To quantitate 4E-BP1 bound to eIF4E, lysates representing 2×10⁶ cells were diluted with buffer A to a final volume of 100 μl and mixed with 25 μl of packed 7-methyl-GTP agarose beads (Pharmacia Biotechnology Inc.) that were pre-equilibrated with buffer B (50 mM Tris, pH 7.5, 150 mM NaCl). Each lysate was mixed with beads in a microcentrifuge tube for 1.5 h at 40° under constant gentle agitation, followed by a brief centrifugation to pellet the beads. The pelleted beads with bound material were washed twice with 0.5 ml of buffer B and eluted with 50 μl of buffer C (25 mM Tris, pH 7.5; 75 mM NaCl; 70 μM 7-methyl-GTP). The 50 μl of eluate were removed completely from the beads, subjected to SDS-PAGE and transferred to nitrocellulose for immunoblotting.

### EXAMPLE 7

### Expression of the eIF-4E Sequestering Agent 4E-BP1 Stimulates Apoptotic Death in Naturally Occurring Cancer Cells

Major limiting factors in anti-neoplastic therapy are the failure of some tumor types to respond to anticancer treatments, and the appearance of resistant cell populations in originally responsive malignancies upon relapse. It is widely recognized that most cytotoxic antineoplastic therapies do not kill cells by causing catastrophic damage to critical structures, but rather by triggering intrinsic apoptotic pathways (reviewed by Kaufmann and Gores, 2000). There is a large body of evidence showing that tumor cells harbor genetic changes leading to increased synthesis of a limited set of proteins which are encoded by messenger RNAs containing specific elements in their 5′ and 3′ untranslated regions (reviewed by De Benedetti and Harris, 1999 and Zimmer et al. 2000). These alterations are often accompanied by cancer cell chemoresistance and are thus often associated with a poor prognosis.

Initiation of cap-dependent translation in mammals is positively regulated by the cap-binding protein eIF4E and inhibited by the translational repressor 4E-BP1, which prevents associations between eIF4E and caped mRNAs (reviewed by Sonenberg, 1996; Sonenberg and Gingras, 1998; Raught and Gingras, 1999). It has also been discovered that increased expression of eIF4E rescues drug-induced apoptosis (Tan et al., 2000). As shown above, transfer of the gene encoding 4E-BP1 into malignant cells activates the intrinsic apoptotic machinery, sensitizes these cells to anti-cancer therapy in vitro, and dramatically reduces their tumorigenicity in vivo. Most importantly from a therapeutic point of view, 4E-BP1 specifically activates apoptosis in cancer cells, but not in normal cells, identifying the cap-dependent translational aparatus as a potential novel molecular target for anticancer drug discovery.

Although the studies show that gain and loss of translation initiation activity potently modulates viability in oncogene-transformed rodent fibroblasts, it was of interest to assess whether translational control is relevant to regulation of apoptosis in naturally occurring cancer cells. It was also of interest to assess whether translation control was relevant to the regulation of apoptosis in naturally occurring cancer cells having tumor-related gene alterations. As model systems to assess the relevance of translational control in the regulation of apoptotis, breast cancer cells which are of epithelial origin and posses diverse tumor-related gene alterations, and non-small cell lung cancer cells were chosen.

Breast Carcinoma Cell Lines Express Activation States of Both Apoptotic Machinery and Cap-dependent Translational Apparatus

To detect whether susceptibility to spontaneous and drug-induced apoptosis in different human breast cancer cell lines correlates with activity of the cap-dependent translational machinery, protein drug-induced apoptosis assays were performed in a set of human breast carcinoma cell lines (Table 1), the genetic profile of which have been documented previously (Sepp-Lorensino et al., 1995).

TABLE 1
Cell lines in use in our studies

| | Ras | | Cell |
|---|---|---|---|
| Cell line | status | Other characteristics | line source |
| HMEC 184 A1 | wt | Immortalized breast | Berkeley |
| | | epithelial cells | National |
| | | | Laboratory |
| MDA-MB-231 | Ki-V12 | ER- | ATCC |
| MDA-MB-453 | wt | ER−/erbB2+++/MAPK+++ | ATCC |
| MCF-7 | wt | ER+/IGF-IR +++ | Dr. Yee, |
| | | | UM Cancer |
| | | | Center |
| MDA-MB-468 | wt | ER−/EGFR+++ | Dr. Yee, |
| | | | UM Cancer |
| | | | Center |
| SkBr-3 | wt | ER−/HER2+++ | Dr. Yee, |
| | | | UM Cancer |
| | | | Center |

The Ras status, estrogen (ER) dependence, and other characteristics of non-transformed breast epithelial cells and breast carcinoma lines are shown. One cancer cell line (MDA-MD-231) harbors mutated Ki-Ras, while others express activated upstream effectors of eIF4E signaling pathways.

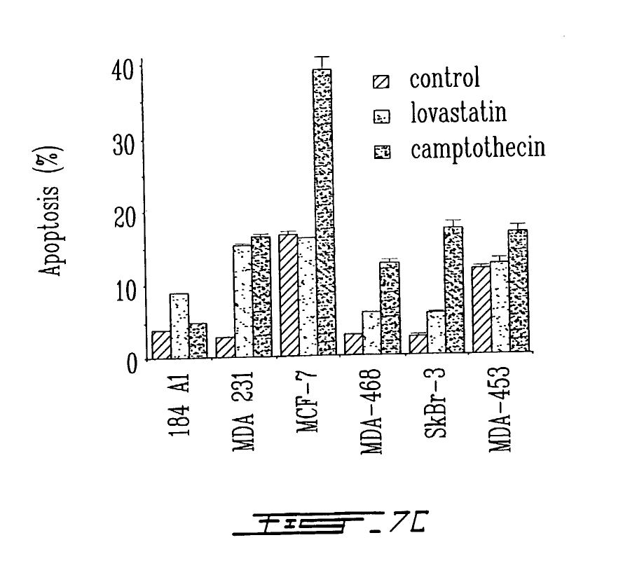

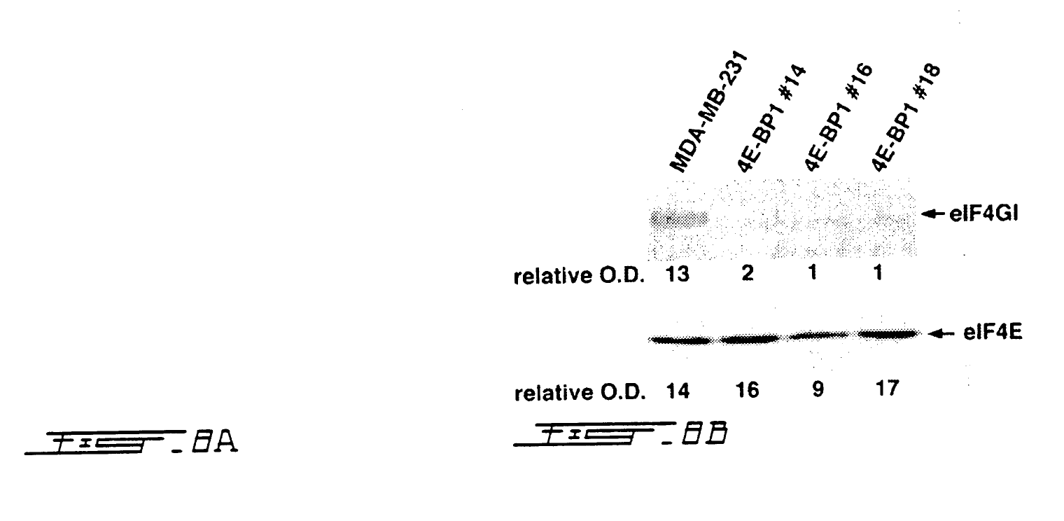

Extracts from non-transformed breast epithelial cells and breast cancer cells lines were tested to detect cellular levels of eIF4E and 4E-BP1, and to evaluate the association of eIF4GI with eIF4E to form an intact translation initiation complex (FIG. 7). Steady state levels of eIF4E were similar among the breast cancer cell lines and modestly increased compared to the non-transformed 184 A1 breast epithelial cells (FIG. 7a). Steady state levels of eIF4G1 were significantly increased in all breast cancer cell lines tested. While 4E-BP1 is predominantly represented in non-transformed cells by hypophosphorylated isoform a which actively represses translation, breast cancer cell extracts are enriched for slow migrating hyperphosphorylated 4E-BP1 (isoforms β and γ) which is much less active in repressing assembly of eIF4F. Consistent with this, breast cancer cell extracts manifested increased amounts of eIF4GI associated with eIF4E in the cap bound fraction (FIG. 7b). This indicates increased amounts of intact eIF4F complex in all breast cancer cell lines tested, suggesting these cells function in a translationally activated state.

Apoptosis assays revealed elevated spontaneous apoptosis in MCF-7 and, MDA-MB453 cells as well as increased susceptibility to drug-induced apoptosis in all tested breast carcinomas (FIG. 7c). Since upregulated cap-dependent translation antagonizes apoptotic death as described above and in Polunovsky et al., 1996; and in Tan et al., 2000, it was hypothesized that breast cancer cells require a high level of cap-dependent translation to suppress the apoptotic apparatus that is activated in the course of cell malignant transformation.

Enforced Expression of 4E-BP1 Stimulates Spontaneous and Drug-induced Apoptosis in MDA-MB-231 Breast Carcinoma.

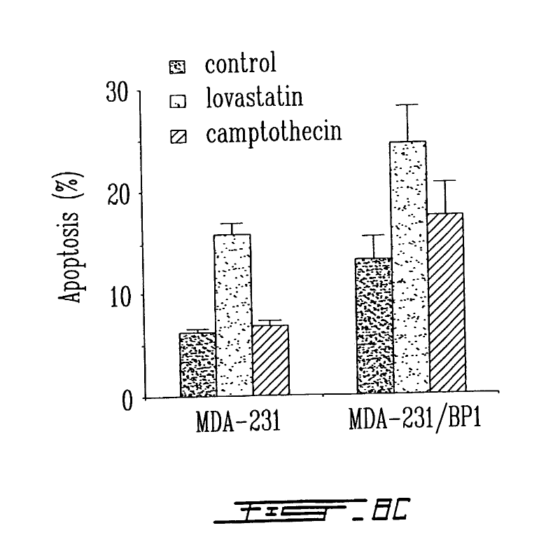

To test the hypothesis that breast cancer cells are dependent or an increase in cap-dependent translation to overcome the apoptosis pathway, the effects of inhibitors of cap-dependent translation on spontaneous and drug-induced apoptosis in normal and malignant cells were examined. Since breast cancer cells significantly differ from normal epithelial cells in expression of 4E-BP1 (FIG. 7A), the focus was put first on developing cell lines in which cap-dependent translation is inhibited by enforced overexpression of 4E-BP1. As shown above, the translational repressor, and eIF4E-[sequestering agent] 4E-BP1 and 4E-BP1 phosphorylation-inhibitor rapamycin, sensitized transformed fibroblasts to apoptosis and suppresses Ras-dependent tumorigenicity in a manner strictly dependent on their ability to sequester eIF4E from a translationally active complex with eIF4G. To determine whether the anti-apoptotic effect of 4E-BP1 observed in Ras-transformed fibroblasts is also seen in naturally occurring cancer cells, breast cancer cells, MDA-MB-231 cells expressing oncogenic Ras, were stably transfected with a neo selectable vector pACTAG-2 engineered to encode haemagglutinin (HA) tagged human wt 4E-BP1(Gingras et al., 1999). Twelve neomycin-resistant clones were isolated and assayed for steady state expression of HA; subjected to cap-affinity chromatography to quantify the proportion of eIF4E complexed with eIF4GI and examined for chemosensitivity to lovastatin and camptothecin (FIG. 8). In the three clones shown (FIG. 8a), HA expression (as a surrogate for ectopic 4E-BP1), eIF4E captured by cap-analog, and eIF4GI associated with cap-bound eIF4E can be compared to these parameters in non-transfected MDA-MB-231 cells. Ectopic 4E-BP1 displaced eIF4GI from eIF4E (FIG. 8b). This was accompanied by an increase in intrinsic apoptosis and substantially augmented apoptotic death in response to the lovastatin or camptothecin (FIG. 8c).

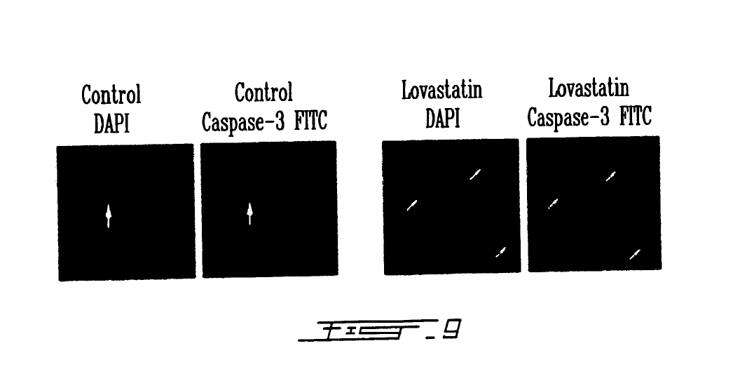

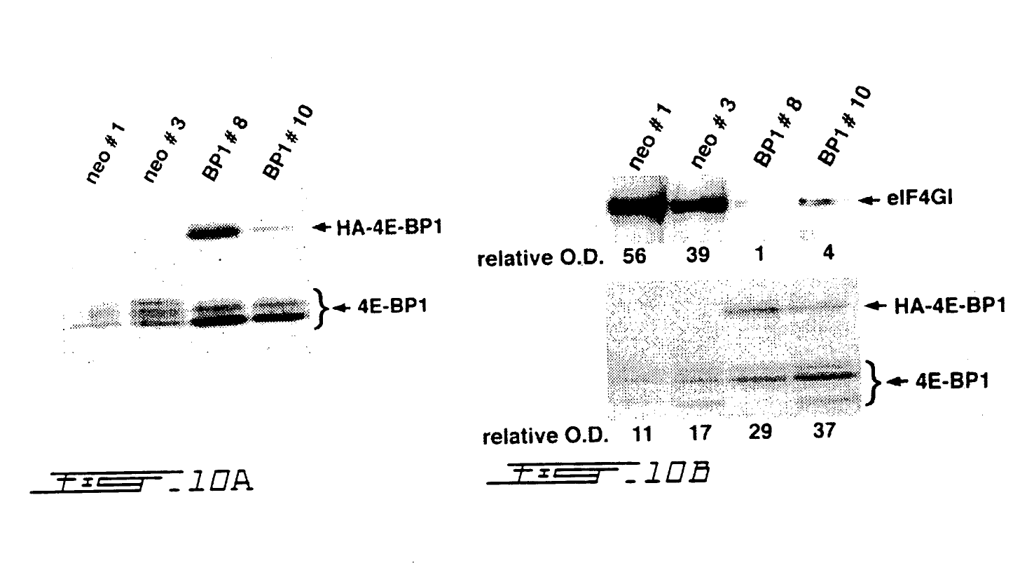

To confirm activation of apoptosis-related biochemical events in 4E-BP1 expressing cells and independently verify the results of flow cytometry, immunomorphological techniques in which nuclear apoptotic rearrangements are identified along with expression of active capspase-3 were used (FIG. 9).

Ectopic Expression of 4E-BP1 Activates Apoptosis in Non-small Cell Lung Cancer (NSCLC) Cells.

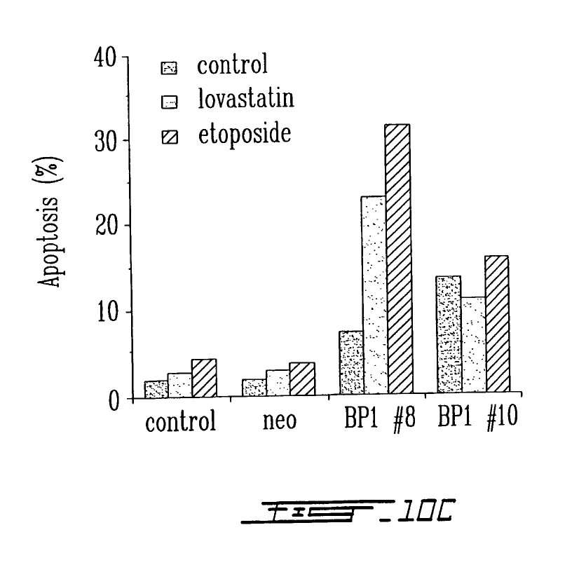

To further validate that the present invention is relevant to the clinical situation and more specifically to validate that translation control is involved in the modulation of apoptosis in naturally occurring cancer cells, the NSCLC cell line 2009 harboring Ki-RasV12 was stably transfected with the neo vector carrying HA-tagged 4E-BP1 or with empty vector. Two HA-4E-BP1 positive and two mock-transfected clonal cell lines were subjected to cap-affinity chromatography to detect expression levels of slow migrating HA-4E-BP1, eIF4GI captured by the cap-complexed eIF4E and apoptotic response. Cells expressing high levels of exogenous HA-4E-BP1 (clone #8), manifested significant suppression of the eIF4G1/eIF4E/cap complex formation and were highly susceptible to the pro-apoptotic effect of both lovastatin and etoposide (FIG. 10). Clone #10 (a mid-level expressor) displayed intermediate values, while mock-transfected and control cells had a minimal response.

Taken together, the present data validates the hypothesis that levels of expression of the translational factor eIF4E determine chemoresistance in human breast and lung cancer cell lines. Indeed, it was found that both the apoptotic and translational machinery are activated in all tested breast carcinomas when compared to non-transformed breast epithelial cells. Together with the data presented above that activated cap-dependent translation can rescue cells from apoptotic death, these findings demonstrate the proof of principle that in naturally occurring cells which have acquired metabolic alterations leading to increased cap-dependent translation to oppose transformation-related activation of their intrinsic apoptotic program. In addition, ectopic expression of wild type 4E-BP1 stimulates apoptosis and abrogates chemoresistance in breast carcinoma cells expressing oncogenic Ras and in clonal lung cancer cell lines. Activation of apoptosis by translational inhibitors parallels their ability to disrupt eIF4E/eIF4G assembly and repress function of the cap-dependent translation apparatus.

These results suggest that the integrity of the cap-dependent translational apparatus is critically important for viability and chemoresistance in naturally occurring cancer cell. They also demonstrate that targeted disruption of the cap-binding complex by transferring the 4E-BP1 gene can be used as a novel approach to block malignant progression in carcinomas and/or tumors whose growth depends on an activation of cap-dependent translation and more particularly in breast or lung carcinoma. The present invention thus opens the way to the use of preclinical models for cancer in which there is an activation of cap-dependent translation (e.g. breast and lung cancer) to test the ability of upregulated 4E-BP1 to collaborate with well-tolerated doses of available cancer therapeutics to inhibit xenograft growth in athymic mice.

### Conclusion

These data identify a novel survival pathway from Ras through FRAP to 4E-BP1-inhibitable translation initiation, providing new insights into the biology of cancer. Many cancers and tumor cell lines have increased levels of eIF4E (25). Ectopic eIF4E transforms immortal fibroblasts and cooperates with myc in the transformation of primary fibroblasts, whereas reduction of eIF4E level (26) or suppression of eIF4E function by overexpressed 4E-BP1 (21) reverts Ras-transformed cells to a non-malignant phenotype. Available data, therefore, suggest that eIF4E is a powerful oncogene and that 4E-BP1 has the potential to function as a tumor suppressor gene. Although further studies are required to discover the downstream effectors of the eIF4E/4E-BP1 regulated survival pathway and to delineate how they interact with the apoptotic apparatus, the present findings add translational control to the established transcriptional and post-translational mechanisms that regulate cell viability downstream of Ras. More broadly, the present findings add translational control to the established transcriptional and post-translational mechanisms that regulate apoptosis in cells. The inatant invention therefore has broad implications to all diseases or conditions in which a perturbation of apoptosis is encountered.

The findings that ectopic expression of 4E-BP1 selectively kills and chemosensitizes Ras-transformed cells have clear implications for cancer therapeutics. Non-malignant cells can apparently function normally over a wide range of 4E-BP1 expression. Its absence in knockout mice results in no apparent phenotype (27). Moreover, it is herein shown that even a dramatic overexpression in non-transformed fibroblasts is compatible with normal physiological function. However, against the background of oncogenic Ras, 4E-BP1 exerts a powerful control over growth and viability. Taken together, these findings suggest that translational repressors may constitute a significant component of the mammalian tumor surveillance system, and that they might be safely augmented for cancer treatment. Of equal therapeutic importance, since overexpressed eIF4E has been detected in many tumors and malignant cell lines (25), the present invention suggests a novel mechanism whereby tumor cells can acquire resistance to genotoxic and non-genotoxic anticancer agents. Many properties of eIF4E and 4E-BP1, including their ability to regulate proliferation, apoptosis and drug resistance make them potential therapeutic targets in human malignancy.

Although the present invention has been described hereinabove by way of preferred embodiments thereof, it can be modified, without departing from the spirit and nature of the subject invention as defined in the appended claims.

References

1. C. Y. Chenn et al., J. Biol. Chem. 273, 16700 (1998); J. Downward, Curr. Opin. Gen. Dev. 8, 49 (1998); A. B. Vojtek and C. J. Ders, J. Biol. Chem 273, 19925 (1998).

2. A. Kauffman-Zeh et al., Nature 385, 544 (1997).

3. P. Rodriquez-Viciana et at., Nature 370, 527 (1994); P. Rodriquez-Viciana et al., EMBO J. 15, 527 (1994).

4. M. W. Mayo et al., Science 278, 1812 (1997); C. Beraud et al., Biochemistry 96, 429 (1999).

5. J. Downward, Curr. Opin. Cell Biol. 10, 262 (1998).

6. R. S. Datta et al., Cell 91, 231 (1997); L. Peso et al., Science 278, 687.(1997); M. H. Cardone et al., ibid. 282, 1318 (1998).

7. A. Brunet et al., Cell 96, 857 (1999).

8. A. C. Gingras et al., Genes & Dev. 12, 502 (1997).

9. R. Yao and G. M. Cooper, Oncogene 13, 343 (1996); C. Minshall et al., J. Immunology 162, 4542 (1999).

10. T. M. Kinoshita et al., Oncogene 15, 619 (1997).

11. D. Rousseau et al., Cancer Res. 9, 1982 (1995).

12. J. F. Boylan et al., Anticancer Res. 10, 717(1990).

13. J. W. Choi and S. E. Jung, J. Pharm. Exp. Therap. 289, 572 (1999).

14. R. M. Frederickson et al., Mol. Cell. Biol. 12, 1239 (1992); C. W. Rinker-Schaeffer et al., J. Biol. Chem. 267, 10659 (1992).

15. N. Sonenberg, In Translational Control (Cold Spring Harbor Laboratory Press, Cold Spring Harbor, N.Y., ed. J. W. Hershey, M. B. Mathews, and N. Sonenberg, 1996), pp. 245-269. N. Sonenberg and A. C. Gingras, Curr. Opin. Cell. Biol. 10, 268 (1998); A. C. Gingras et al., Ann. Rev. Biochem. 68, 913 (1999).

16. B. Raught and A. C. Gingras, Int. J. Biochem. Cell Biol. 31, 43 (1999).

17. A. Lazaris-Karatzas et al., Nature 345, 544 (1990); A. Lazaris-Karatzas et al., Mol. Cell. Biol. 12, 1234 (1992).

18. Polunovsky et al., Mol. Cell. Biol. 16, 6573 (1996).

19. T. A. Lin et al., Science 266, 653 (1994); A. Pause et al., Nature 371, 762 (1994).

20. A. Haghighat et al., EMBO J. 14, 5701 (1995); P. Fadden et al., J. Biol. Chem. 272, 10240 (1997); S. Maderet al., Mol. Cell. Biol. 15, 4990 (1995).

21. D. Rousseau et al., Oncogene 13, 2425 (1996); L. Beretta et al., EMBO J. 15, 658 (1996); G. J. Brunn et al., J. Biol. Chem. 272 (51), 32547 (1997); J. C. Lawrence Jr. and R. T. Abraham et al., TIBS 22, 345 (1997).

24. J. Marcotrigianao et al., Mol. Cell 3, (1999, in press).

25. A. De Benedetti and A. L. Harris, Int. J. Biochem. and Cell Biol. 31, 59 (1999); I. B. Rosenwald et al., Oncogene 18, 2507 (1999).

26. C. W. Rinker-Shaeffer et al., Int. J. Cancer 55, 841 (1993); J. R. Graff et al., Int. J. Cancer 60, 255 (1995).

27. P. J. Blackshear et al., J. Biol. Chem. 272 (50), 31510 (1997).

28. Marcotrigiano et al., 1999, Molecul. Cell 3:707-716.

Gingras A C, Gygi S P, Raught B. Polakiewicz R D, Abraham R T, Hoekstra M F. Aebersold R. and Sonenberg N. Regulation of 4E-BP1 phosphorylation: a novel two-step mechanism. Genes & Dev 1999; 13:1422-37.

Kaufmann S H and Gores G J. Apoptosis in cancer: cause and cure. BioEssays. 2000; 22: 107-1017.

Sepp-Lorensino L, Ma Z, Rands E, Kohl N E, Gibbs J B, Oliff A, and Rosen N. A peptidomimetic inhibitor of farnesyl:protein transferase blocks the anchorage-dependent and-independent growth of human tumor cell lines. Cancer Res 1995. 5302-5309.

Tan A, Bitterman P, Sonenberg N, Peterson M, and Polunovsky V. Inhibition of Myc-dependent apoptosis by eukaryotic translation initiation factor 4E requires cyclin D1. Oncogene 2000, 19: 1437-1447.

Zimmer S G, De Benedetti A, and Graff J. Translation control of malignancy: the mRNA cap-binding protein, eIF4E, as a central regulator of tumor formation, growth, invasion, and metastasis. Anticancer Res 2000, 20: 1343-1352

What is claimed is:

1. A method of inducing apoptosis in a cell in which the apoptotis pathway is inhibited, which comprises decreasing the amount of eIF4F pre-initiation complex, releaving an apoptotis block.

2. The method of claim 1, wherein said cell is a transformed cell.

3. The method of claim 2, wherein said transformed cell is a Ras-transformed cell.

4. The method according to one of claim 1 to 3, wherein said decrease in the amount of eIF4F pre-initiation complex involves a sequestration of eIF4E in a complex with an eIF4E sequestering agent.

5. The method of claim 4, wherein said eIF4E sequestering agent comprises a sequence having amino acid sequence +φφY-+xfLφxxRxSP or sequence +φxYx+xfLφxxxxxx, wherein x is any amino acid.

6. The method of claim 5, wherein said sequestering agent is selected from 4E-BP, eIF4G, P82, P150, P130 and P20.

7. The method of claim 5, wherein said sequestering agent is selected from 4E-BP1, 4E-BP2, 4E-BP3, eIF4G1, eIF4G2, 4E-BP, and eIF4G.

8. The method of claim 6, wherein said sequestering agent is selected from mammalian 4E-BP1, mammalian 4E-BP2, mammalian 4E-BP3, mammalian eIF4G1 and mammalian eIF4G2.

9. The method of claim 8, wherein said mammalian sequestering agent is a human eIF4E sequestering agent.

10. The method according to one of claims 3 to 9, comprising a transfection of said Ras-transformed cell with a vector encoding eIF4E-sequestering agent or a fragment thereof comprising an eIF4E interaction domain.

11. The method according to one of claims 3 to 9, comprising a transfection of said Ras-transformed cell with a vector encoding 4E-BP1 or a fragment thereof comprising an eIF4E interaction domain.

12. A method of reversing an oncogene-induced chemoresistance in transformed cell which comprises decreasing the amount of eIF4F pre-initiation complex, thereby decreasing the translation of mRNAs implicated in oncogene-induced chemoresistance.

13. The method of claim 12, wherein said oncogene-induced chemoresistance is a Ras-induced chemoresistance.

14. The method according to claim 12 or 13, wherein said decrease in the amount of eIF4F pre-initiation complex, involves a sequestration of eIF4E in a complex with an eIF4E sequestering agent.

15. The method according to claim 12, 13, or 14, wherein said eIF4E sequestering agent is 4E-BP1.

16. The method according to claim 13, comprising a transfection of said Ras-transformed cell with a vector encoding 4E-BP1 or a fragment thereof comprising an eIF4E interaction domain.

17. A method of selecting a compound that modulates direct binding of 4E-BP1 with eIF4E comprising the steps of:
(a) incubating a candidate compound with a first polypeptide comprising a 4E-BP1 interaction domain and a second polypeptide comprising an eIF4E interaction domain under conditions wherein said 4E-BP1 interaction domain binds to said eIF4E interaction domain in the absence of said candidate compound; and
(b) determining the amount of a binding complex formed between said first polypeptide and said second polypeptide, wherein a potential modulating compound is selected when an amount of said binding complex formed in the presence of said candidate compound is measurably different than in the absence thereof.

18. A method of selecting a compound that increases or potentiates a pro-apoptotic function of 4E-BP1 in a cell in which apoptotic function is blocked or inhibited, comprising the steps of:
a) incubating a candidate compound with a first polypeptide comprising a 4E-BP1 interaction domain and a second polypeptide comprising an eIF4E interaction domain and cap structure binding domain, under conditions wherein said 4E-BP1 interaction domain binds to said eIF4E interaction domain in the absence of said candidate compound; and
b) determining the amount of a binding complex between said first polypeptide and said second polypeptide, using a cap affinity column to capture said second polypeptide through its cap structure binding site and associated binding partners, wherein a compound that potentiates or increases the pro-apoptotic function of 4E-BP1 is selected when an amount of said binding complex formed between said first and second polypeptide in the presence of said candidate compound is measurably different than in the absence thereof.

19. The method of claim 18, wherein said cell is a Ras-transformed cell.

20. A method of immortalizing an oncogene-induced transformed cell which comprises increasing the amount of eIF4F pre-initiation complex, thereby increasing the translation of mRNAs implicated in the oncogene-induced anti-apoptotic pathway.

21. The method of claim 20, wherein said oncogene is Ras.

22. The method according to claim 20, wherein said increase in the amount of eIF4F pre-initiation complex involves desequestration and/or an inhibition of the sequestration of eIF4E in a complex with an eIF4E sequestering agent.

23. The method according to claim 20, wherein said eIF4E sequestering agent is 4E-BP1.

24. The method of claim 22, wherein said desequestration or said inhibition of sequestration is effected by an antibody specific to the eIF4E interaction domain of 4E-BP1, or the epitope-bearing portion thereof.

25. The method of claim 22, wherein said desequestration or said inhibition of said sequestration is effected by an inhibition of the synthesis of 4E-BP1.

26. The method of claim 25, comprising an agent which inhibits the synthesis of 4E-BP1, wherein said agent comprises an antisense RNA complementary to the nucleotide sequence encoding for 4E-BP1.

27. The method of claim 25, wherein said inhibition of synthesis is dependent on an antisense RNA complementary to 4E-BP1.

28. Use of an eIF4E sequestering agent, 4E-BP1 or an eIF4E binding portion thereof for treating high proliferative cells.

29. A method of treating cancer comprising a treatment of oncogene-induced cancer cells with a combination of chemotherapeutic agent and an agent which decreases the amount of active pre-initiation complex.

30. The method of claim 29, wherein said oncogene is Ras.
